(19) United States
(12) Patent Application Publication
(10) Pub. No.: US 2024/0342138 A1
(43) Pub. Date: Oct. 17, 2024

(54) MICROTUBULE TARGETING AGENTS

(71) Applicant: BPGbio, Inc., Framingham, MA (US)

(72) Inventors: Vivek K. VISHNUDAS, Chelmsford, MA (US); Maria-Dorothea Nastke, Holliston, MA (US); Stephane Gesta, Concord, MA (US)

(21) Appl. No.: 18/635,905

(22) Filed: Apr. 15, 2024

Related U.S. Application Data
(60) Provisional application No. 63/525,377, filed on Jul. 7, 2023.
(60) Provisional application No. 63/459,173, filed on Apr. 13, 2023.

(51) Int. Cl. A61K 31/4196; A61P 35/00

(52) U.S. Cl. CPC A61K 31/4196; A61P 35/00

(57) ABSTRACT

Provided are methods for treating cancers responsive to the modulation of microtubule assembly and P-gp-mediated resistant cancers using 2-(difluoromethoxy)-N-[[5-(2-methoxyphenyl)-1H-1,2,4-triazol-3-yl]methyl]benzamide, or a pharmaceutically acceptable salt thereof.

### RELATED APPLICATIONS

This application claims priority to U.S. Provisional Patent Application No. 63/525,377 filed on Jul. 7, 2023, and U.S. Provisional Patent Application No. 63/459,173 filed on Apr. 13, 2023, the contents of each of which are incorporated herein in their entirety.

### BACKGROUND

Microtubule-targeting agents (MTAs) are among the most effective chemotherapeutics used in the treatment of cancer. However, the clinical utility of current MTAs, such as the microtubule-targeting vinca alkaloids (e.g. vinblastine and vincristine) and taxanes (paclitaxel and docetaxel), are often limited due to adverse side effects or multidrug resistance (MDR). Previous studies have established that broad-based resistance to these drugs, arises in large part from the overexpression of P-glycoprotein (P-gp), to which paclitaxel, vinblastine, vincristine, docetaxel, and others, are substrates. See e.g., Gottesman et al. (2002), Nat Rev Cancer 2 48-58 and Mol Pharmacol. 2009 January; 75(1): 92-100. This presents major challenges to cancer chemotherapy, particularly in managing patients with metastatic cancers that are resistant to traditional MTA therapies.

To overcome P-gp-mediated MDR, many small molecule drugs which modulate the activity of P-gp have been tested. See e.g., Darby et al., Curr. Drug. Metab 2011, 12, 722-731. Most of these programs, however, have failed clinical trials due to lack of potency and/or toxicity issues. The need therefore exists for novel microtubule interactors, particularly those which are not substrates for P-gp.

### SUMMARY

Evidence now suggests that 2-(difluoromethoxy)-N-[[5-(2-methoxyphenyl)-1H-1,2,4-triazol-3-yl]methyl]benzamide, herein referenced to as Compound 1, inhibits the formation of microtubule assembly. See e.g., FIG. 1.

Provided herein, therefore, are methods of treating a cancer responsive to the modulation of microtubule assembly using 2-(difluoromethoxy)-N-[[5-(2-methoxyphenyl)-1H-1,2,4-triazol-3-yl]methyl]benzamide, or a pharmaceutically acceptable salt thereof. Such cancers include e.g., prostate cancer, head and neck cancer, endometrial cancer, glioblastoma multiforme, sarcoma, and lung cancer.

Evidence also shows that Compound 1 is not a substrate for P-gp, the overexpression of which leads to P-gp-mediated multi-drug resistance with certain vinca alkaloids and taxane chemotherapeutics. Therefore, also provided are methods of treating P-gp-mediated resistant cancers using Compound 1, or a pharmaceutically acceptable salt thereof.

In certain aspects, the disclosure relates to a method of treating a cancer responsive to the modulation of microtubule assembly comprising administering to a subject in need thereof, a therapeutically effective amount of 2-(difluoromethoxy)-N-[[5-(2-methoxyphenyl)-1H-1,2,4-triazol-3-yl]methyl]benzamide, or a pharmaceutically acceptable salt thereof. In some embodiments, the cancer is selected from prostate cancer, head and neck cancer, endometrial cancer, glioblastoma multiforme, sarcoma, and lung cancer. In some embodiments, the cancer is prostate cancer. In some embodiments, the cancer is head and neck cancer. In some embodiments, the cancer is lung cancer. In some embodiments, the lung cancer is small cell lung cancer (SCLC) or non-small cell lung cancer (NSCLC). In some embodiments, the cancer is endometrial cancer. In some embodiments, the cancer is glioblastoma multiforme. In some embodiments, the cancer is sarcoma.

In certain aspects, the disclosure relates to a method of treating a P-gp-mediated resistant cancer comprising administering to a subject in need thereof, a therapeutically effective amount of 2-(difluoromethoxy)-N-[[5-(2-methoxyphenyl)-1H-1,2,4-triazol-3-yl]methyl]benzamide, or a pharmaceutically acceptable salt thereof. In some embodiments, the cancer is a taxane resistant cancer. In some embodiments, the cancer is resistant to paclitaxel, vinblastine, vincristine, or docetaxel. In some embodiments, the cancer is resistant to paclitaxel. In some embodiments, the cancer is selected from ovarian, prostate, breast, bladder, head and neck, and lung cancer. In some embodiments, the cancer is ovarian cancer. In some embodiments, the cancer is breast cancer. In some embodiments, the cancer is bladder cancer. In some embodiments, the cancer is head and neck cancer. In some embodiments, the cancer is lung cancer. In some embodiments, the cancer is a vinca alkaloid resistant cancer. In some embodiments, the cancer is resistant to vinblastine or vincristine. In some embodiments, the cancer is selected from lymphoma, acute lymphocytic leukemia (ALL), and solid tumors.

### BRIEF DESCRIPTION OF THE FIGURES

FIG. 1 shows the Vmax values for Compound 1, paclitaxel, and the positive control nocodazole in a microtubule polymerization assay.

FIG. 2 is a western blot supporting that Compound 1 binds to the colchicine binding site of tubulin.

FIG. 3 shows the significance in the upper band intensity (relative to total B-tubulin) from the Western blot in FIG. 2.

FIG. 4 illustrates the effect on kinetochore assembly of HCT116 cells treated with Compound 1.

FIG. 5 illustrates the effect on kinetochore assembly of HCT116 cells treated with Compound 1.

FIG. 6 shows the spindle assembly checkpoint activation of HCT116 cells treated with Compound 1.

FIG. 7A-7D shows the potency of Compound 1 in patient-derived organoids (PDOs) representative of multiple tumor types. PDOs were treated with Compound 1 or controls (vehicle control—0.1% DMSO, positive control—10% DMSO) for 5 days. Viability was assessed using the CellTiter-Glo® assay. IC50 and maximal (max) inhibition values were extrapolated from the viability curves. Exemplars of models deemed to be highly responsive (>60% max inhibition; A), responsive (40-60% max inhibition; B), and non-responsive (<40% max inhibition; C) to Compound 1 are shown. Summary of all models tested (D); NR (No Regression, ambiguous nonlinear regression curves or R2<0.65) and NA (Not Applicable, ambiguous maximum inhibition). Compiled data of N=4 replicates.

FIG. 8 shows tissue distribution results at various time points resulting from oral administration of Compound 1 to SD rats.

FIGS. 9A and 9B show potency of Compound 1 in patient-derived organoid (PDO) models of taxane resistance. Patient-derived organoids were treated with Compound 1, paclitaxel or controls (0.1% DMSO, positive control—10% DMSO) for 5 days. Viability was assessed using the CellTiter-Glo® assay. IC50 and maximal (max) inhibition values were extrapolated from the viability curves. A) Model overview and dose response curves to Compound 1 and paclitaxel. B) Summary of CellTiter-Glo results in all three models tested. Compiled data of N=4 replicates.

FIG. 10A-10C show potency of Compound 1 in a patient-derived organoid model of taxane resistance. A) Overview of study design. B) CTG-1520 was implanted subcutaneously in nude mice and tumors were allowed to grow to a volume of 210 mm³ followed by randomization of animals into three (3) groups (vehicle control, 75 mg/kg, 150 mg/kg Compound 1) with 12 animals per group. A Compound 1 solid dispersion was prepared prior to each dosing and administered by oral gavage two times a day (BID). Mean tumor volume of each group over time and the growth of tumor in each individual animal was graphed. For each individual animal of vehicle group tumor growth inhibition (TGI) % was calculated based on the tumor growth inhibition of individual tumor at each given day of the study compared to the average of the group for that given day of the study. P-values were calculated using two-tailed distribution and two-sample equal variance (homoscedastic) for each group treated with Compound 1 for each given day of the study in reference to vehicle control. ns p>0.05; *p≤0.05; **p≤0.01. C) Body weight of each individual animal was measured on days 0, 3, 6, 10, 13, and 17 and graphed using Graphpad Prism.

FIG. 11A-11E show the efficacy of Compound 1 in a colorectal cancer xenograft model (COLO205). A) In vitro potency of Compound 1 in COLO205 cells as evaluated by ATPlite 1Step™ Perkin Elmer (performed by NTRC Oncolines). B) COLO205 cells were implanted subcutaneously in nude mice and tumors were allowed to grow to a volume of 210 mm3 followed by randomization of animals into three (3) groups (vehicle control, 75 mg/kg, 150 mg/kg Compound 1) with 12 animals per group. A Compound 1 solid dispersion was prepared prior to each dosing and administered by oral gavage two times a day (BID). Mean tumor volume of each group over time and the growth of tumor in each individual animal was graphed. For each individual animal of vehicle group TGI (%) was calculated based on the tumor growth inhibition of individual tumor at each given day of the study compared to the average of the group for that given day of the study. P-values were calculated using two-tailed distribution and two-sample equal variance (homoscedastic) for each group treated with Compound 1 for each given day of the study in reference to vehicle control. NS p>0.05; *p≤0.05; **p≤0.01; ***p p≤0.001; **** p≤0.0001. C) Plasma and tumor tissues from vehicle (n=11), 75 mg/kg (n=12), and 150 mg/kg (n=11) groups were harvested at the end of the study. The concentration of Compound 1 in the plasma or tumor tissue was plotted as a column plot (Average t SEM) or Scatter Plot with Median. D) Tumor tissues from vehicle (n=11), 75 mg/kg (n=12) and 150 mg/kg (n=11) groups were harvested at the end of the study when each animal was sacrificed. The level of two biomarkers, pHH3 and CCNB1 in each tissue was measured and graphed as the Min-to-Max plot and scatter plot of the level of each biomarker. The line in the scatter plot indicates the group mean. E) Body weight of each individual animal was measured on each day of the study and graphed using Graphpad Prism.

FIG. 12A-12E show the efficacy of Compound lin a prostate cancer xenograft model (DU145). A) In vitro potency of Compound 1 in DU145 cells as evaluated by ATPlite 1Step™ Perkin Elmer (performed by NTRC Oncolines). B) DU145 cells were implanted subcutaneously in nude mice and tumors were allowed to grow to a volume of 210 mm³ followed by randomization of animals into three (3) groups (vehicle control, 75 mg/kg, 150 mg/kg Compound 1) with 12 animals per group. A Compound 1 solid dispersion was prepared prior to each dosing and administered by oral gavage two times a day (BID). Mean tumor volume of each group over time and the growth of tumor in each individual animal was graphed. For each individual animal of vehicle group TGI (%) was calculated based on the tumor growth inhibition of individual tumor at each given day of the study compared to the average of the group for that given day of the study. P-values were calculated using two-tailed distribution and two-sample equal variance (homoscedastic) for each group treated with Compound 1 for each given day of the study in reference to vehicle control. NS p>0.05; *p≤0.05; **p≤0.01; ***p p≤0.001; **** p≤0.0001. C) Plasma and tumor tissues from vehicle (n=11), 75 mg/kg (n=12), and 150 mg/kg (n=12) groups were harvested at the end of the study. The concentration of Compound lin the plasma or tumor tissue was plotted as a column plot (Average t SEM) or Scatter Plot with Median. D) Tumor tissues from vehicle (n=12), 75 mg/kg (n=12) and 150 mg/kg (n=12) groups were harvested at the end of the study when each animal was sacrificed. The level of two biomarkers, pHH3 and CCNB1 in each tissue was measured and graphed as the Min-to-Max plot and scatter plot of the level of each biomarker. The line in the scatter plot indicates the group mean. E) Body weight of each individual animal was measured on days 0, 2, 5, 7, 9, 12, 14, 16, 19, 21, 23, and 26 and graphed using Graphpad Prism.

FIG. 13A-13B show efficacy of Compound 1 in a lung adenocarcinoma xenograft model (A549). A) In vitro potency of Compound 1 in A549 cells as evaluated by ATPlite 1Step™ Perkin Elmer (performed by NTRC Oncolines). B) A549 cells (5×10⁶) were implanted subcutaneously in athymic female nude mice and tumors were allowed to grow to a volume of ˜112 mm³ followed by randomization of animals into three (3) groups (vehicle control, 75 mg/kg, 150 mg/kg Compound 1) with 12 animals per group. A Compound 1 solid dispersion was prepared prior to each dosing and administered by oral gavage two times a day (BID). Mean tumor volume of each group over time and the growth of tumor in each individual animal was graphed. For each individual animal of vehicle group TGI (%) was calculated based on the tumor growth inhibition of individual tumor at each given day of the study compared to the average of the group for that given day of the study. P-values were calculated using two-tailed distribution and two-sample equal variance (homoscedastic) for each group treated with Compound 1 for each given day of the study in reference to vehicle control. NS p>0.05; *p≤0.05; **p≤0.01; ***p p≤0.001; **** p≤0.0001.

FIG. 14A-14E show the efficacy of Compound 1 in glioblastoma cell lines in vitro and in a glioblastoma rat model (C6). A) In vitro potency of Compound 1 in four different glioblastoma cell lines as evaluated by Realtime-Glo™ assay (C6, and U87 cells) or ATPlite 1Step™ Perkin Elmer (performed by NTRC Oncolines; T98G and A-172 cells). B) Study design overview: C6 rat glioma cells (3×10⁵) were implanted into the right entorhinal cortex/subiculum region of male Sprague Dawley rats and tumor burden was assessed 14 days post inoculation using MRI and animals randomized into three (4) groups (vehicle control, 5 mg/kg, 10 mg/kg, and 20 mg/kg Compound 1) with 4 animals per group. A Compound 1 solid dispersion was prepared prior to each dosing and administered by oral gavage two times a day (BID). C) Body weights were measured every two days. D) Survival curve of vehicle versus treated animals. E) MRI images of long-term survivors at beginning of study (D0), post 16 days of treatment (D17) and post 31 days of treatment.

### DETAILED DESCRIPTION

In certain aspects, the disclosure relates to a method of treating a cancer responsive to the modulation of microtubule assembly comprising administering to a subject in need thereof, a therapeutically effective amount of 2-(difluoromethoxy)-N-[[5-(2-methoxyphenyl)-1H-1,2,4-triazol-3-yl]methyl]benzamide, or a pharmaceutically acceptable salt thereof. The term “cancer responsive to the modulation of microtubule assembly” as used herein refers to a cancer that is responsive to a microtubule-targeting agent, such as a vinca alkaloid (e.g., vinblastine or vincristine) or a taxane (e.g., paclitaxel and docetaxel). In some embodiments, the cancer that is responsive to the modulation of microtubule assembly is selected from the group consisting of prostate cancer, head and neck cancer, endometrial cancer, glioblastoma multiforme, sarcoma, and lung cancer.

In one aspect, provided is a method of treating a cancer selected from prostate cancer, head and neck cancer, endometrial cancer, glioblastoma multiforme, sarcoma, and lung cancer in a subject comprising administering to the subject a therapeutically effective amount of 2-(difluoromethoxy)-N-[[5-(2-methoxyphenyl)-1H-1,2,4-triazol-3-yl]methyl]benzamide, or a pharmaceutically acceptable salt thereof. Also provided is a therapeutically effective amount of 2-(difluoromethoxy)-N-[[5-(2-methoxyphenyl)-1H-1,2,4-triazol-3-yl]methyl]benzamide, or a pharmaceutically acceptable salt thereof, for treating a cancer selected from prostate cancer, head and neck cancer, endometrial cancer, glioblastoma multiforme, sarcoma, and lung cancer. Further provided is the use of a therapeutically effective amount of 2-(difluoromethoxy)-N-[[5-(2-methoxyphenyl)-1H-1,2,4-triazol-3-yl]methyl]benzamide, or a pharmaceutically acceptable salt thereof, in the manufacture of a medicament for treating a cancer selected from prostate cancer, head and neck cancer, endometrial cancer, glioblastoma multiforme, sarcoma, and lung cancer. In one aspect, the cancer treated by the aforementioned method is prostate cancer. In another aspect, the cancer treated by the aforementioned method is head and neck cancer. In yet another aspect, the cancer treated by the aforementioned method is lung cancer such as small cell lung cancer (SCLC) or non-small cell lung cancer (NSCLC). In yet another aspect, the cancer treated by the aforementioned method is endometrial cancer. In yet another aspect, the cancer treated by the aforementioned method is glioblastoma multiforme. In yet another aspect, the cancer treated by the aforementioned method is a sarcoma.

In one aspect, provided is a method of treating a P-glycoprotein (P-gp)-mediated resistant cancer in a subject comprising administering to the subject a therapeutically effective amount of 2-(difluoromethoxy)-N-[[5-(2-methoxyphenyl)-1H-1,2,4-triazol-3-yl]methyl]benzamide, or a pharmaceutically acceptable salt thereof. Also provided is a therapeutically effective amount of 2-(difluoromethoxy)-N-[[5-(2-methoxyphenyl)-1H-1,2,4-triazol-3-yl]methyl]benzamide, or a pharmaceutically acceptable salt thereof, for treating a P-gp-mediated resistant cancer. Further provided is the use of a therapeutically effective amount of 2-(difluoromethoxy)-N-[[5-(2-methoxyphenyl)-1H-1,2,4-triazol-3-yl]methyl]benzamide, or a pharmaceutically acceptable salt thereof, in the manufacture of a medicament for treating a P-glycoprotein (P-gp)-mediated resistant cancer.

In one aspect, the P-gp-mediated resistant cancer described herein is a taxane resistant cancer. In another aspect, the P-gp-mediated resistant cancer described herein is a vinca alkaloid resistant cancer. In some embodiments, the P-gp-mediated resistant cancer described herein is a paclitaxel, vinblastine, vincristine, and/or docetaxel resistant cancer.

As used herein, “P-glycoprotein mediated resistant cancer” or “Pg-p mediated resistant cancer”, used interchangeably herein, refers to a cancer which is resistant to treatment with one or more anticancer agents which are substrates for P-glycoprotein (Pg-p). Such anticancer agents include, but are not limited to, paclitaxel, vinblastine, vincristine, and docetaxel.

The term “resistance”, in the context of a resistant cancer, means that the cancer no longer responds to treatment. This includes cancers which are unresponsive or demonstrate a worsening of the disease while on a given treatment. In one aspect, a resistant cancer refers to a cancer which has become resistant during the course of treatment, i.e., the cancer initially responded, but no longer responded to treatment after a certain period of time.

As used herein, “taxane resistant cancer” refers to a cancer which is resistant to treatment with taxane anticancer agents. Taxanes are known in the art and include small molecules comprising tetradecahydro-6,10-methanobenzo[10]annulene and derivatives thereof as a central core. Taxane resistant cancers can include e.g., ovarian, prostate, breast, bladder, head and neck, and lung cancers.

As used herein, “vinca alkaloid resistant cancer” refers to a cancer which is resistant to treatment with vinca alkaloid anticancer agents. Vinca alkaloids are known in the art and include small molecules comprised of two multi-ringed units, an indole nucleus, and a dihydroindole nucleus, joined together with other complex systems. Vinca alkaloid resistant cancers include e.g., lymphomas, acute lymphocytic leukemia (ALL), and solid tumors.

For use in medicines, the pharmaceutically acceptable salt salts described herein refer to non-toxic “pharmaceutically acceptable salts.” Pharmaceutically acceptable salt forms include pharmaceutically acceptable basic/cationic salts.

The terms “subject” and “patient” may be used interchangeably, and refer to a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the terms “treatment,” “treat,” and “treating” refer to reversing, alleviating, delaying the onset of, or inhibiting the progress of a described cancer. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “effective amount” or “therapeutically effective amount” refers to an amount of 2-(difluoromethoxy)-N-[[5-(2-methoxyphenyl)-1H-1,2,4-triazol-3-yl]methyl]benzamide that will elicit a biological or medical response of a subject e.g., a dosage of between 0.01-100 mg/kg body weight/day. In one aspect, the effective amount of Compound 1 ranges from about 50 mg/kg to about 250 mg/kg. In one aspect, the effective amount of Compound 1 is about 75 mg/kg, about 100 mg/kg, about 150 mg/kg or about 200 mg/kg.

In some aspects, Compound 1 may be administered as part of a pharmaceutical composition. Pharmaceutical compositions may be administered orally, parenterally, by inhalation spray, topically, rectally, nasally, buccally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. In some embodiments, the compositions are administered orally. Sterile injectable forms of the compositions described herein may be aqueous or oleaginous suspension. These suspensions may be formulated according to techniques known in the art using suitable dispersing or wetting agents and suspending agents.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

### EXAMPLES

2-(difluoromethoxy)-N-[[5-(2-methoxyphenyl)-1H-1,2,4-triazol-3-yl]methyl]benzamide (Compound 1) has the chemical structure shown below and may be synthesized according to the procedures described for compound 126 in U.S. Pat. No. 11,091,447, the entire contents of which are incorporated herein by reference.

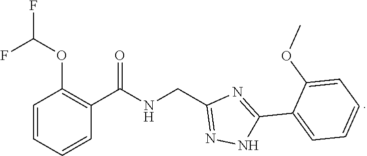 [SMILES: COc1ccccc1-c1nc(CNC(=O)c2ccccc2OC(F)F)n[nH]1]

2-(difluoromethoxy)-N-[[5-(2-methoxyphenyl)-1H-1,2,4-triazol-3-yl]methyl]benzamide may exist in various tautomeric forms, each of which are expressly included as part of the invention.

### Example 1. Effect of Compound 1 on Tubulin Polymerization

In vitro tubulin polymerization assays (using >99% Pure Tubulin, OD Based—Porcine (BK006P) from Cytoskeleton Inc.) demonstrated that certain anti-cancer effects from Compound 1 are a result of cytoskeleton targeting to inhibit microtubule assembly.

### Methods

The protocol followed for tubulin polymerization is according to the manufacturer's protocol. The following components were diluted as follows:
A vial of lyophilized GTP was diluted in 100 μl of sterile water and made into 10 μl aliquots and stored at −80° C.
Tubulin was diluted to 10 mg/ml in buffers+GTP as recommended, flash frozen in liquid nitrogen and stored at −80° C.
Paclitaxel was diluted to 2 mM in DMSO

The half area 96-well plate provided by kit manufacturer was allowed to warm up to 37° C. for 30 mins in the Molecular Devices M5 plate reader. The Compound 1 and nocodazole were diluted in DMSO to a concentration of 2 mM, followed by dilution of these working stocks in the General Tubulin Buffer (GTB-provided in the kit) to a 10× the final well concentration and final well volume of 10 μl. Same was done with the 2 mM Paclitaxel stock. Once the plate was warmed up, the compounds were added in duplicates to the wells. In the control wells (no ligand control) 10 μl of GTB was added. The plate was placed at 37° C. in the plate reader for 3 minutes. Flash frozen tubulin was diluted as follows to have a final concentration of 10% glycerol and 1 mM GTP:
For 3 mg/ml tubulin: 200 μl of reconstituted tubulin+420 μl of Tubulin polymerization buffer (750 μl GTB+250 μl Tubulin glycerol buffer+10 μl 100 mM GTP)
For 2 mg/ml tubulin: 200 μl of reconstituted tubulin+800 μl of Tubulin polymerization buffer (950 μl GTB+250 μl Tubulin glycerol buffer+12 μl 100 mM GTP)
100 μl of the diluted tubulin was added to each of the wells containing warmed compounds and the experiment was started immediately. The experimental setup was as follows:
   Absorbance read: 340 nm
   Temperature: 37° C.
   Shake: 5 s at medium speed prior to first read.
Read: The absorbance was read every 1 min for 90 minutes (total 91 reads).

The values were normalized to the first read by designating the first read as ‘0’ subtracting the value of the first read from the following reads.

### Results

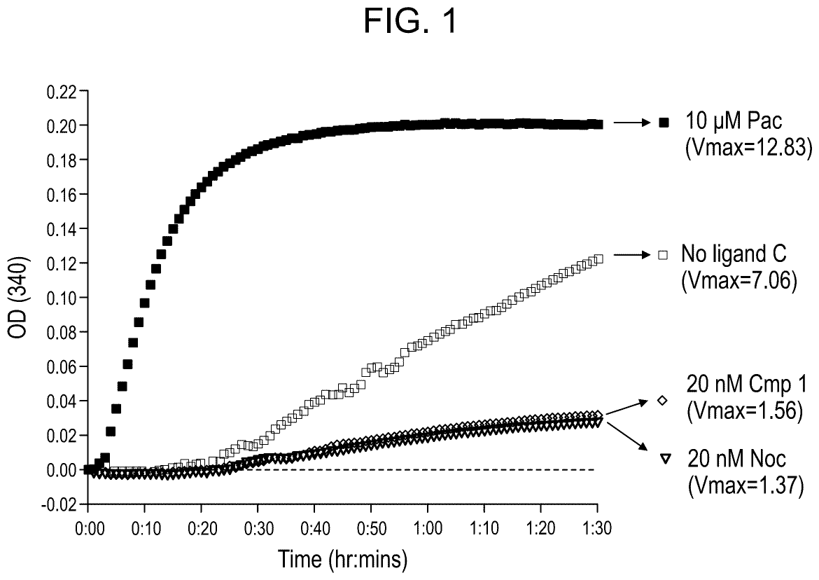

Microtubule polymerization, as a readout of absorbance at 340 nm, under standard conditions (no ligand control) and in the presence of 10 μM paclitaxel, a microtubule polymerization agent, as well has high (10 μM) and low (20 nM) concentrations of Compound 1 and positive control nocodazole are shown with their respective Vmax values in FIG. 1. Representative data plots of N=3 replicates. As shown, Compound 1 inhibits microtubule polymerization and to a similar extent as nocodazole in the assay.

### Example 2. Binding of Compound 1 to Colchicine Binding Site of Tubulin

### Materials

MIA PaCa2 cells
DMEM growth medium (Gibco, cat #1195-065, lot #2366044)
Fetal Bovine Serum (FBS)
Penicillin/Streptomycin
DMSO
RIPA lysis buffer
BRG519, Compound 1, Colchicine, Vinblastine, Paclitaxel and Nocodazole stocks (1000× or 2000×)
EBI (N,N′-ethylene-bis(iodoacetamide))
PBS
Halt protease inhibitor cocktail (Thermo Fisher, catalogue number #87786)

### Methods

### Cell Culture:

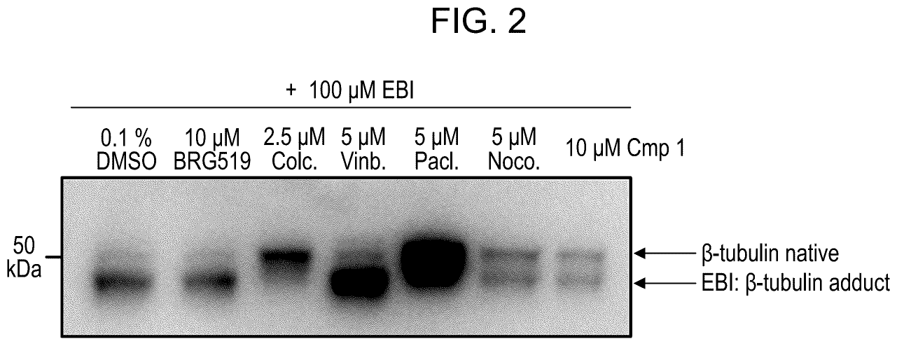

MIA PaCa2 cells were cultured in DMEM+10% FBS and 1% P/S. Cells were plated at 1.5-2×10⁶ in 60 mm dishes. For drug treatments, cells were treated the day after plating with 0.1% DMSO or the compounds tested at the concentrations indicated in FIG. 2 for 2 hours.

### EBI Labeling Assay

Cells were treated with compounds for 2 hours at 37°, 5% CO₂ (in the cell culture incubator), and then for an additional 2 hours with 100 μM EBI (with compounds present) under the same conditions.
Cells were then harvested with Trypsin and pelleted at 500 g for 3 minutes.
Cells were rinsed in 1 mL PBS and transferred to clean epi tubes. Cells were pelleted again at 500 g for 3 minutes and then lysed in 300 μL RIPA+Halt protease inhibitor.
Cell lysates were sonicated for 10 seconds each on 20% power and kept on ice afterwards.
BCA assay was performed to normalize protein concentration to reduce the intensity of the paclitaxel bands (these bands were always a lot brighter than those in other samples, consistent between replicates and troubleshooting samples). BCA assay plates were incubated for 15 minutes in the 37-degree incubator in the outer molecular lab.
After protein normalization (including addition of reducing Laemmli buffer from a 6× stock) samples were boiled at 95 degrees for 3 minutes and frozen for subsequent Western Blotting.

Significance in difference of treated MIA PaCa-2 cells to 0.1% DMSO treated cells were tested by Student t test (* p<0.05; ** p<0.01; *** p<0.001).

### Results

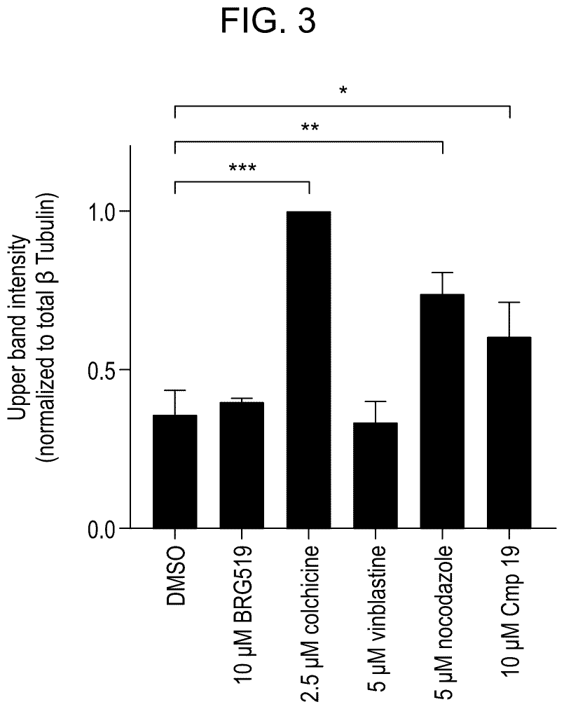

As shown in FIG. 2 and FIG. 3, Compound 1 inhibited the formation of EBI (N,N′-ethylene-bis(iodoacetamide)):β-tubulin adducts indicating an occupied colchicine-binding site of tubulin. Nocodazole and colchicine also inhibited formation of the EBI:β-tubulin adducts in the assay.

### Example 3. Kinetochore Assembly of Cells Treated with Compound 1

### Materials

HCT116 cells
DMEM cell growth media (Thermofisher CAT #11995073; Lot #1930072)
Fetal Bovine Serum: Sigma Aldrich,
Trypsin-EDTA Solution (IX): Gibco, #25300096 (100 ml)
Penicillin/Streptomycin: Gibco, #15140-122 (100 ml)
Poly-D-Lysine German Glass Coverslips (#1.5, 18 mm) (Electron Microscopy sciences #72294-04)
Dulbecco's Phosphate Buffered Saline (1×) w/o Calcium and Magnesium (Fisher #BW17-512Q)
Mad1 antibody (GeneTex #GTX105079)
α-Tubulin antibody (Cell Signaling #3873S)
Paraformaldehyde 8% Aqueous solution (Electron Microscopy Sciences (#157-8)
Goat Serum (Novus Biologicals #NBP2-23475)
Alexa Fluor® 594 AffiniPure Donkey Anti-Human IgG (Jackson Immunoresearch #709-585-149)
Alexa Fluor® 488 AffiniPure Donkey Anti-Mouse IgG (Jackson Immunoresearch #709-585-150)
Triton™ X-100 (molecular bio. Grade; Sigma #T8787)
ProLong Gold Anti-Fade Mount with DAPI (Fisher Scientific #S36938)

### Methods

### Cell Culturing:

The HCT116 cells were cultured using DMEM media supplemented with 10% FBS and 1% Pen/Strep. Cells were maintained to a maximum of 80% confluency before being split and plated into coverslips in 12-well plates.

Antibody List

| Antibody | Dilution |
|---|---|
| α-Tubulin | 1:1000 |
| Mad1 | 1:1000 |
| Goat anti-human 546 | 1:1000 |
| Goat anti-mouse 488 | 1:1000 |

### Immunocytochemistry:

1. HCT116 cells were plated on Poly-D-Lysine German Glass Coverslips (#1.5, 18 mm)

2. After 24h cells were treated with 300 nM of Compound 1 and Nocodozole.

3. After 24h cells were washed briefly with PBS and fixed with warm 4% paraformaldehyde at room temperature for 10 min.

4. Cells were permeabilized and blocked in 10% goat serum, 0.5% Triton-X in PBS for 1 hr.

5. Cells were incubated with Primary antibodies overnight.

6. Washed 3× with PBS for 10 min

7. Incubated with secondary antibodies at room temperature for 1 hr.

8. Washed 3× with PBS for 10 min

9. Coverslips were mounted with ProLong Gold Anti-Fade Mount with DAPI and imaged with Olympus FV1200 MPE Microscope.

10. Images were collected with identical confocal settings for all of the samples and Z-stacked images were projected with maximal projection mode using Fluor View Software.

### Calculation the Corrected Total Cell Fluorescence (CTCF) for Mad1 Using ImageJ.

1. Selected cells of interest using the drawing/selection tools (i.e. rectangle, circle, polygon or freeform).

2. From the Analyze menu selected “Measure.”

3. Selected region with no fluorescence to be my background.

4. Repeated these steps for other cells in the field of view

5. Used the CTCF formula to calculate Mad1 expression:

CTCF=Integrated Density−(Area of selected cell×Mean fluorescence of background readings)

Micrographs were imaged using Olympus FV1200 MPE confocal microscope at 63× oil. White arrows indicate dividing cells. Representative images of N=3 biological replicates. Significance in difference of treated HCT116 cells to untreated cells was tested by Student t test (** p<0.01).

### Results

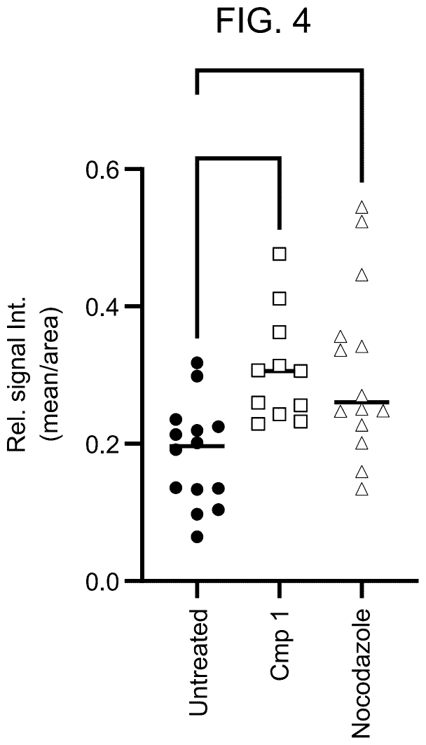

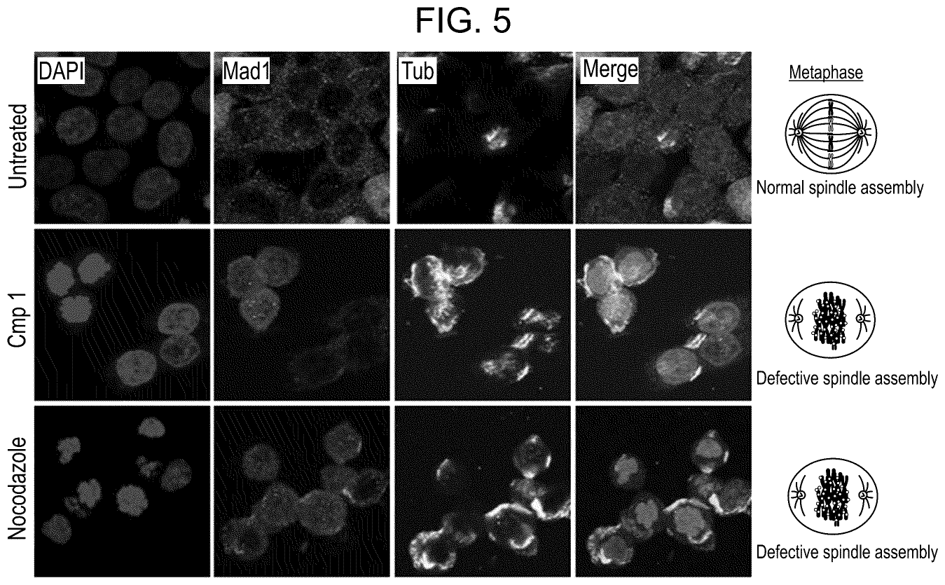

As shown in FIG. 4, confocal microscopic evaluation of kinetochore assembly of cells treated with Compound 1 or nocodazole revealed a lack of proper mitotic spindle formation (A), highlighted with an increase in Mad1 signal intensity (B) emphasizing metaphase arrest. See also FIG. 5.

### Example 4. Spindle Assembly Checkpoint Activation

### Materials

HCT116 cell line
DMEM Lonza #12-604F, Lot #0001008586
PBS: Lonza #17-512Q Lot #02204
FBS: Gibco #26140-087 Lot #2206642RP
Trypsin EDTA Gibco cat 25200-056 Lot #2323073
Penicillin/Streptomycin Gibco #14140-122 lot #02204
Compound 1
Nocodazole: MedChemExpress: #HY-13520 Lot 10555
Ro-3306: MedChemExpress: #HY-12529 Lot #14923
Hesperadin, MedChemExpress: #HY-12054 Lot #08617
DMSO: Invirogen #D12345
Bovine Serum Albumin, Boston Bioproducts (#P-753), lot: 21CN127
Triton X-100: Sigma Cat: T8787-100 mL Lot: SLC06163
CS&T beads: BD biosciences #661415 lot 1182462
EDTA Invitrogen #15575-038 Lot 2393343
Ethyl alcohol, Pure 200 proof, for molecular biology (Sigma-Aldrich, CAT #E7023)
PBT buffer (PBS, 1% BSA, 0.25% TritonX100)
PBE buffer (PBS, 1% BSA, 2 mM EDTA)
Alexa Fluor® 488 anti histone H3 phospho (Ser10) Ab (Biolegend CAT #650804)
Alexa Fluor® 488 Mouse IgG2b, κ Isotype Ctrl Antibody (Biolegend CAT #400329)
7AAD (BD Pharmingen CAT #559925)
Accuri C6 Plus flow cytometer

### Methods

1. Hct116 were grown in DMEM with 10% FBS and 1% Penicillin/Streptomycin.
2. Cells were plated into 6 well dishes at indicated cell numbers below (i.e. plate 1=300 k cells per well) and treatment started later the same day. Asynchronous (Asynch) control cells (Plate 1 were collected 1 day after plating. Compound 1 and Nocodazole were both 300 nM, CDK1i Ro-3306 10 μM, Aurora Bi (ABi) Hesperadin 50 μM.
3. Cells were treated with Compound 1 or Noc for 20 hours then sequentially without changing media they were treated with CDK1i or ABi for 4 more hours. Alternatively, cells were treated with Compound 1 or Noc for 20 h then washed twice with warm media and grown in fresh media for 4 hours to allow passage through mitosis into G1 as diploid cells.
4. Note that plate 4 treatments were in general extremely toxic to cells or inconclusive.

### Flow Cytometry Collection and Staining:

1. Collected all media and cells into the same conical tube. Centrifuged cells at 1000×g for 3 min.
2. Aspirated off supernatant, washed with PBS.
3. Centrifuged cells as above.
4. Resuspended cells in cold 400 μl and while gently vortexing added 1 mL cold 100% ethanol slowly dropwise (fixes and permeabilizes)
5. Stored at 4 degrees C. for at a minimum of 1 hour, or up to one week.
6. Antibody staining:
   First washed 1× with PBT (PBT buffer (PBS, 1% BSA, 0.25% TritonX100), and aspirated supernatant.
   Finally, labeled with 7AAD and pH3-488:
   Added 1 ml of PBT, mix, pellet, remove supernatant. Make master mix 100 μl per sample in PBT:
      90 μl PBT
      5 μl α phospho Histone H3-Alexa488 (1:25)
      7.5 μl 7AAD
For isotype samples used 5 μl isotype control
Added to cells, incubated 1 hour room temperature in the dark.
Post incubation, cells were washed 1× with PBT and 1× with PBE and resuspended in 600 μl of PFE.
7. Cells were subsequently subjected to flow cytometric analysis on Accuri C6 Plus, run at relatively slow rate around 200 single cell events per second.
8. Collected around 10,000 single cell events.
9. Analyzed data with FlowJo 10 software and made graphs with GraphPad Prism.

### Results

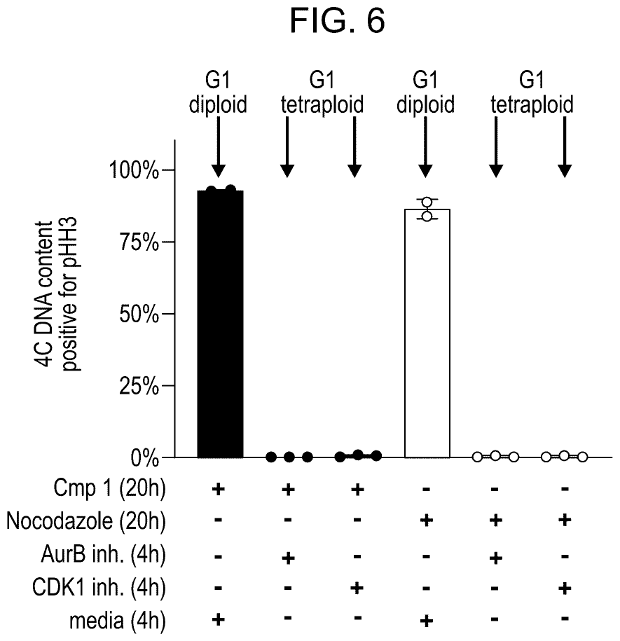

As shown in FIG. 6, a mechanism at or before microtubule-kinetochore attachment in M phase of the cell cycle with Compound 1 was evidenced.

### Example 5. Potency of Compound 1 in Multiple Tumor Types of Patient-Derived Organoids (PDOs)

### Methods

Potency of Compound 1 was evaluated in a panel of patient-derived organoids (PDOs) representative of multiple tumor types.

### Patient-Derived TumorGraft Models

| | Patient | |
|---|---|---|
| # | Model # | Tumor Type |
| 1 | CTG-2647 | Endometrial |
| 2 | CTG-1252 | SCLC |
| 3 | CTG-1094 | Sarcoma |
| 4 | CTG-3303 | Breast |
| 5 | CTG-1932 | NSCLC |
| 6 | CTG-0868 | Ovarian |
| 7 | CTG-0198 | SCLC |
| 8 | CTG-0012 | Breast |
| 9 | CTG-0980 | Sarcoma |
| 10 | CTG-1017 | Breast |
| 11 | CTG-1313 | Sarcoma |
| 12 | CTG-1015 | Glioblastoma multiforme |
| 13 | CTG-0199 | SCLC |
| 14 | CTG-1180 | Ovarian |
| 15 | CTG-2268 | Endometrial |
| 16 | CTG-1106 | Breast |
| 17 | CTG-2713 | Glioblastoma multiforme |
| 18 | CTG-3167 | Prostate |
| 19 | CTG-1367 | Renal cell carcinoma |
| 20 | CTG-0824 | Renal cell carcinoma |
| 21 | CTG-1280 | Endometrial |
| 22 | CTG-2428 | Prostate |
| 23 | CTG-2427 | Prostate |
| 24 | CTG-1520 | Breast |
| 25 | CTG-1868 | Gastric |
| 26 | CTG-1602 | Ovarian |
| 27 | CTG-0064 | Colorectal |
| 28 | CTG-0081 | Colorectal |
| 29 | CTG-1643 | Pancreatic |
| 30 | CTG-2540 | NSCLC |
| 31 | CTG-1366 | Renal cell carcinoma |
| 32 | CTG-0667 | Glioblastoma multiforme |
| 33 | CTG-0439 | Colorectal |
| 34 | CTG-0252 | Ovarian |

### Test Agent Information

Test article: Test articles were prepared fresh weekly.

| | | | | | Working |
|---|---|---|---|---|---|
| | Master Stock | Master Stock | | Working | Stock |
| Agent | Storage | Stability | State | Stock Storage | Stability |
| Compound 1 | −20° C. | 3 months | Solution | −20° C. | Fresh mix |
| Paclitaxel | −20° C. | Per | Solution | −80° C. | 2 years |
| | | Manufacturer | | | |

The vehicle for Compound 1 and Paclitaxel was DMSO.

### Experimental Design

1. Cryopreserved samples (200-300 mg or more depending on study design) were prepared as described below.
2. The collected tumors were dissociated manually (using a tumor mincing process previously developed) or using the Gentle MACS Dissociator and human Tumor Dissociation Kit (Miltenyi). After preparation, tumor fragments were cultured in DMEM+10% FBS+1× antibiotic/antimycotic (“anti-anti”) plus 1:500 Primocin antimicrobial agent for up to 7 days before filtering out large fragments (using a 500 μm filter followed by 200 μm filters).
3. Flow-through was cultured in: Cell-Tak coated low volume flat-bottom 384-well assay plates.
4. 10× concentrations of test agent (or DMSO as vehicle) was added to media in volumes that will vary depending on culturing conditions.
5. Each group had quadruplicate wells, including 0.1% DMSO (final as vehicle) and 10% DMSO as positive control.
6. PBS was plated in the perimeter wells to avoid edge-effect artifacts.
7. Cells were treated for 5 days and viability read via Cell Titer Glo. Viability was quantitatively assessed via Cell Titer Glo at Day 0 (baseline), Day 6 (baseline), and Day 6 (compound treatment-full dose response). Cell Titer Glo data were supplemented with a live dye imaging palette (Hoechst, Cell Tracker Green, MitoTracker Red CMXRos, Draq7) for positive and negative controls as well as 2 doses of test agents (high dose and mid-range dose).

### Results

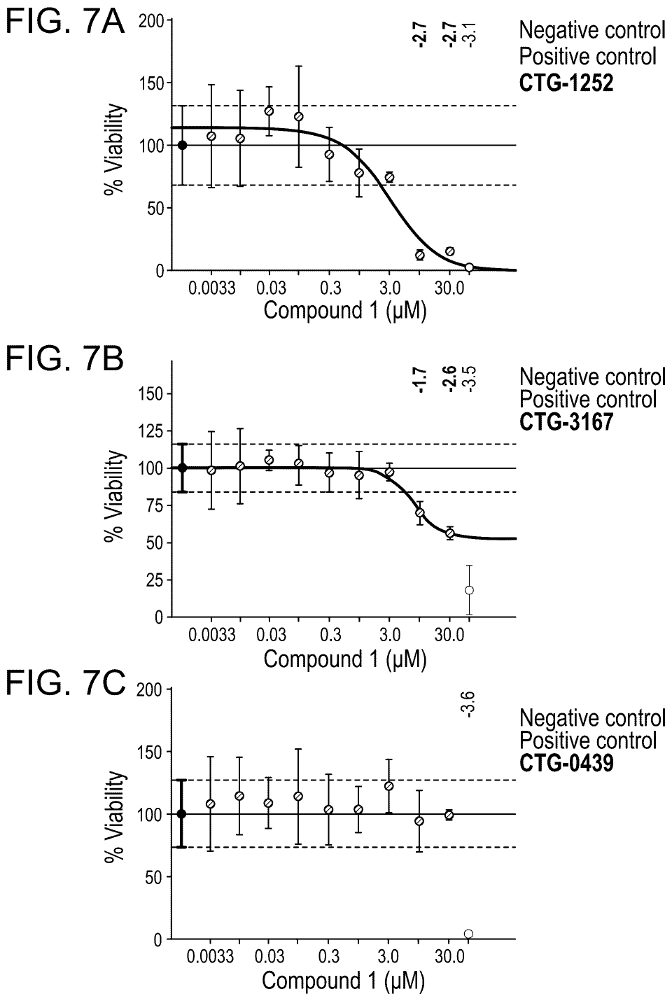

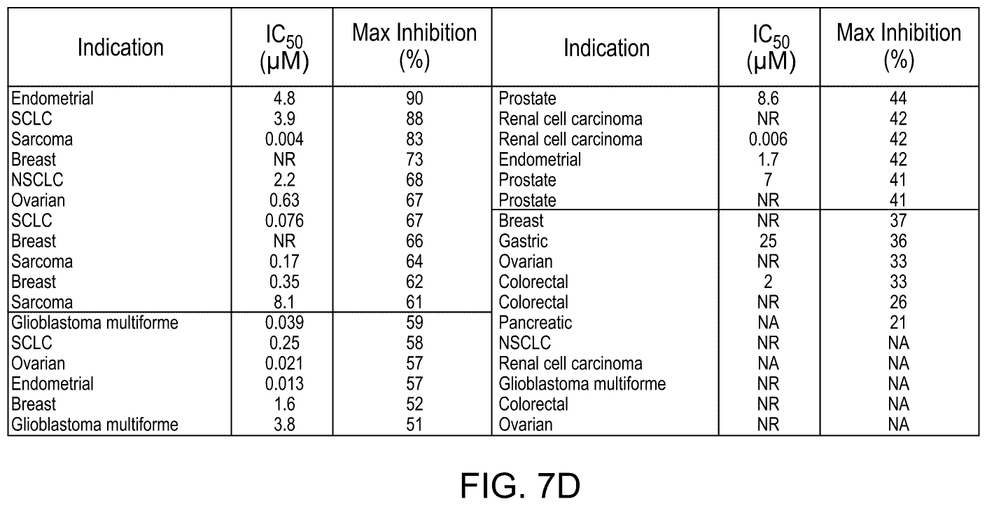

FIGS. 7A-7C show examples of viability curves from PDO models which were highly responsive (FIG. 7A, SCLC), responsive (FIG. 7B, prostate cancer), and non-responsive (FIG. 7C, colorectal cancer) to Compound 1. Cancers deemed to be highly responsive (>60% max inhibition), responsive (40-60% max inhibition), or non-responsive (<40% max inhibition) to Compound 1 are shown in FIG. 7D. For example, tumor samples from patients with endometrial cancer, small cell lung cancer (SCLC), sarcoma, breast cancer, non-small cell lung cancer (NSCLC), and ovarian cancer were shown to be highly responsive to Compound 1. Tumor samples from patients with Glioblastoma multiforme, SCLC, ovarian cancer, endometrial cancer, breast cancer, prostate cancer, or renal cell carcinoma were found to be responsive to Compound 1. Summary of all models tested with highly responsive models highlighted in green, responsive models in blue, and non-responsive models in white (FIG. 7D). Compiled data of N=4 replicates. NR=No Regression; NA=Not Applicable

### Example 6. Biodistribution of Compound 1 in Rats

### Methods

Male Sprague-Dawley Rats (total of 6 animals) 6 to 8 weeks of age (210-227 g) was used in this biodistribution study. Compound 1 was dosed at 75 mg/kg to all animals through oral route of administration at a 10 ml/kg dose volume. Compound 1 formulation was freshly prepared in Tween-80+0.5% methyl cellulose and maintained at room temperature until dosing. Dose was administered in all animals under fasted conditions. Two rats were sacrificed at 0.25 hrs, 1 hr and 8 hrs respectively. Heart, lung, liver, kidney, stomach, small intestine, large intestine, skeletal muscle, brain, spleen, pancreas and adipose were harvested while blood was processed to plasma for bioanalytical quantification of Compound 1.

Compound 1 bioanalytical assay was developed and validated using the SCIEX 6500 LC-MS/MS. A mobile phase (isocratic flow) comprised of Acetonitrile and 0.2% formic acid in MilliQ water in a Atlantis dC18-HPLC column (50×4.6 mm, 3 uM), with a flow rate of 0.9 mL/min and a run time of 2.20 min. Mass transition (m/z) of 375.040 and 188.100 was used for detection of Compound 1 at an LLOQ of 2.14 ng/mL.

### Results

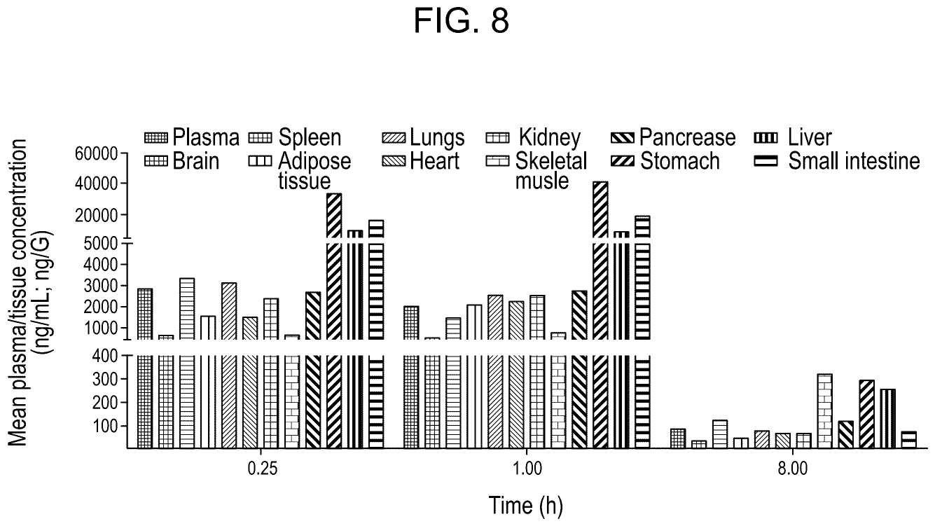

Results are shown below in Table 1-3 and represented graphically by FIG. 8. Drug distribution was revealed across all tissues with pharmacologically significant blood

brain barrier penetration observed at 8 hours.

TABLE 1
Tissue Concentrations (ng/mL or
ng/g) of Compound 1 at 0.25 hours

| | Mean plasma/tissue | Ratio | Percentage |
|---|---|---|---|
| | concentration | (tissue | (plasma |
| Tissue | (ng/mL or ng/g) | to plasma) | to tissue) |
| Plasma | 2832 | NA | 3.59 |
| Brain | 624 | 0.22 | 0.79 |
| Spleen | 3359 | 1.19 | 4.26 |
| Adipose tissue | 1529 | 0.54 | 1.94 |
| Lungs | 3088 | 1.09 | 3.91 |
| Heart | 1489 | 0.53 | 1.89 |
| Kidney | 2362 | 0.83 | 2.99 |
| Skeletal Muscle | 607 | 0.21 | 0.77 |
| Pancreas | 2698 | 0.95 | 3.42 |
| Stomach | 33713 | 11.9 | 42.7 |
| Liver | 9893 | 3.49 | 12.5 |
| Small intestine | 16724 | 5.90 | 21.9 |

TABLE 2
Tissue Concentrations (ng/mL or ng/g) of Compound 1 at 2 hours

| | Mean plasma/tissue | Ratio | Percentage |
|---|---|---|---|
| | concentration | (tissue | (plasma |
| Tissue | (ng/mL or ng/g) | to plasma) | to tissue) |
| Plasma | 1976 | NA | 2.29 |
| Brain | 494 | 0.25 | 0.57 |
| Spleen | 1449 | 0.73 | 1.68 |
| Adipose tissue | 2034 | 1.03 | 2.36 |
| Lungs | 2523 | 1.28 | 2.93 |
| Heart | 2227 | 1.13 | 2.59 |
| Kidney | 2517 | 1.27 | 2.92 |
| Skeletal Muscle | 734 | 0.37 | 0.85 |
| Pancreas | 2738 | 1.39 | 3.18 |
| Stomach | 41462 | 21.0 | 48.1 |
| Liver | 9139 | 4.63 | 10.6 |
| Small intestine | 18820 | 9.53 | 21.9 |

TABLE 3
Tissue Concentrations (ng/mL or ng/g) of Compound 1 at 8 hours

| | Mean plasma/tissue | Ratio | Percentage |
|---|---|---|---|
| | concentration | (tissue | (plasma |
| Tissue | (ng/mL or ng/g) | to plasma) | to tissue) |
| Plasma | 83.1 | NA | 5.37 |
| Brain | 33.6 | 0.40 | 2.18 |
| Spleen | 121.2 | 1.46 | 7.84 |
| Adipose tissue | 47.2 | 0.57 | 3.06 |
| Lungs | 78.2 | 0.94 | 5.06 |
| Heart | 64.8 | 0.78 | 4.19 |
| Kidney | 64.2 | 0.77 | 4.16 |
| Skeletal Muscle | 316 | 3.81 | 20.5 |
| Pancreas | 117 | 1.41 | 7.60 |
| Stomach | 290 | 3.5 | 18.8 |
| Liver | 255 | 3.06 | 16.5 |
| Small intestine | 74.4 | 0.90 | 4.82 |

### Example 7. Blood Brain Barrier Permeability of Compound 1

### Methods

Preparation of Donor Solution a) 0.2 mM working solution was prepared by diluting 10 mM stock solution with DMSO. b) 10 μM donor solution (5% DMSO) was prepared by diluting 20 μL of working solution with 380 μL PBS. 2) 150 μL of 10 μM donor solutions to each well of the donor plate, whose PVDF membrane was precoated with 5 μL of 1% brain polar lipid extract (Porcine)/dodecane mixture. Duplicates were prepared. 3) 300 μL of PBS was added to each well of the PTFE acceptor plate. 4) The donor plate and acceptor plate were combined together and incubated for 4h at room temperature with shaking at 300 rpm. 5) Preparation of TO sample: 20 μL donor solution was transferred to new well followed by the addition of 250 μL PBS (DF: 13.5), 130 μL ACN (containing internal standard) as TO sample. 6) Preparation of acceptor sample: The plate was removed from incubator. 270 μL solution was transferred from each acceptor well and mixed with 130 μL ACN (containing internal standard) as acceptor sample. 7) Preparation of donor sample: μL solution was transferred from each donor well and mixed with 250 μL PBS (DF: 13.5), 130 μL ACN (containing internal standard) as donor sample. 8) Acceptor samples and donor samples were all analyzed by LC/MS/MS. 9) The equation used to determine permeability rates (Pe) was displayed as follow.

$$\\ {P}_{e} = C \times ( - ln ( 1 - \frac{{[ drug ]}_{acceptor}}{{[ drug ]}_{equilibrium}} ) ) \times {10}^{{}_{} 7} , where$$

$$\\ C = ( \frac{{V}_{D} \times {V}_{A}}{( {V}_{D} + {V}_{A} ) \times Area \times time} )$$

$$\\ [ drug ] equilibrium = ( [ drug ] donor \times VD + [ drug ] acceptor \times VA ) / ( VD + VA )$$

$$\\ VD = 0.15 mL ; VA = 0.3 mL ; Area = 0.28 {cm}^{2} ; time = 14400 s .$$

$$\\ [ drug ] acceptor = ( Aa / Ai \times DF ) acceptor ; [ drug ] donor = ( Aa / Ai * DF ) donor ;$$

$$\\ Aa / Ai : Peak area ratio of analyte and internal standard ;$$

$$\\ DF : Dilution factor .$$

### Results

As shown by the results in Table 4, Compound 1 is highly permeable across the blood brain barrier.

TABLE 4

| | Test | | | | |
|---|---|---|---|---|---|
| | Concen- | Incubation | Mean Pe | % Mean | |
| Drug | tration | Time | (nm/s) | recovery | Permeability |
| Atenolol | 10 μM | 4 h | <0.0346 | <100.3 | Low |
| Propranolol | 10 μM | 4 h | 47.4 | 27.7 | High |
| Compound 1 | 10 μM | 4 h | 67.6 | 85.8 | High |

### Example 8. Compound 1 Anti-Tumor Activity in Ex Vivo PDO Models and In Vivo Xenograft Models of Taxane Resistant Breast Cancer

### Methods

Although taxane resistance can be derived in vitro, this approach has limitations, as it does not faithfully recapitulate all aspects of taxane resistance observed in patients. Hence, a study designed to test Compound 1 efficacy in patient-derived organoids (PDO) ex vivo models of taxane resistant cancers was performed. CTG-1520 and CTG-0896 were derived from Triple Negative Breast Cancer (TNBC) patients post-treatment, and progression on a taxane-based therapy and the models' resistance to paclitaxel was confirmed by testing in vivo PDX (Patient-derived xenograft) tumor growth. CTG-1017 was derived from the same patient as CTG-1520 although at an earlier point in the treatment course, and has been shown to be sensitive to paclitaxel in vivo (CRO historical data). Compound 1 and paclitaxel were tested head-to-head in each model in a dose-curve setting. 10% DMSO was used as the positive control for induction of 100% cell cytotoxicity.

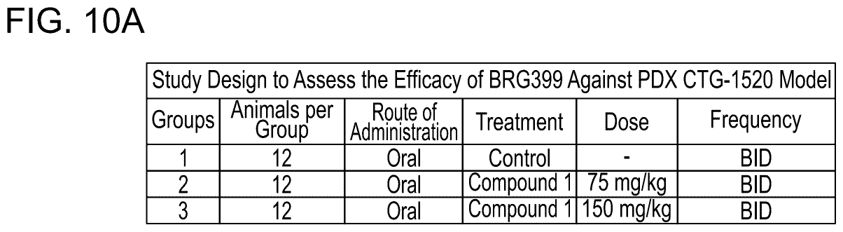

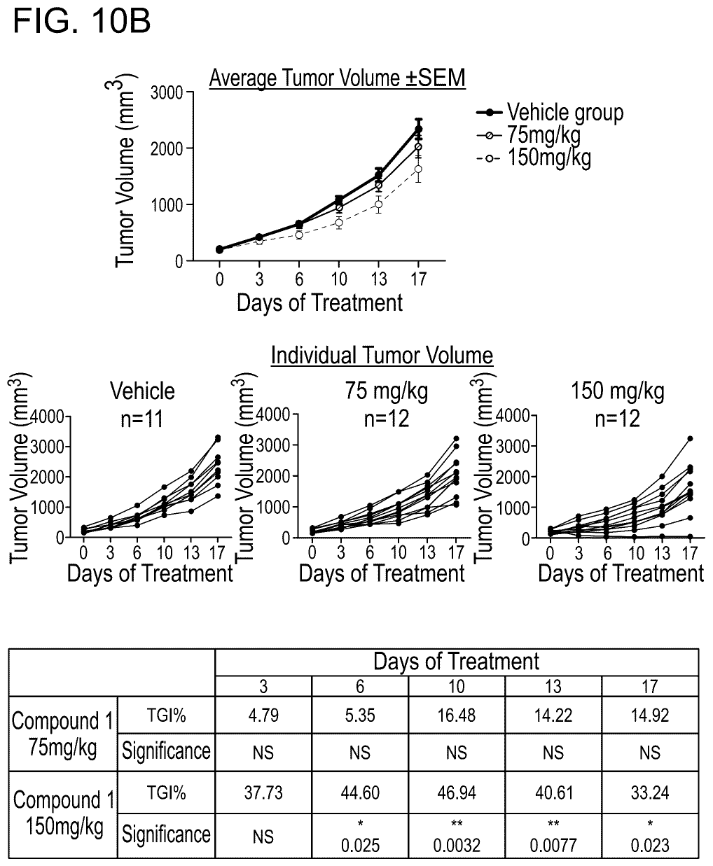

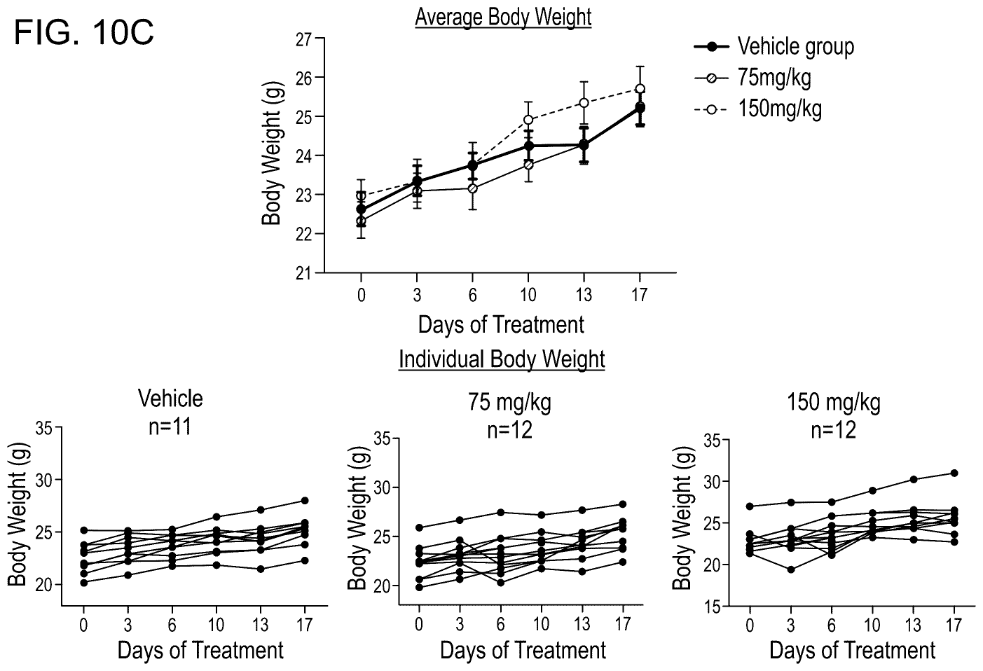

In a next step, the ability of Compound 1 to inhibit the growth of CTG-1520 PDX in nude mice was assessed. CTG-1520 was implanted subcutaneously in nude mice and tumors were allowed to grow to a volume of 210 mm³ followed by randomization of animals into three (3) groups (vehicle control; 75 mg/kg, and 150 mg/kg Compound 1) with 12 animals per group (see FIG. 10A). A Compound 1 solid dispersion was administered by oral gavage two times a day (BID). All animals, except one (1) from the vehicle control group, survived through the study (17 days). One (1) animal from the vehicle control group reached the endpoint of 2000 mm³ prior to day 17 and was eliminated from all calculations.

### Results

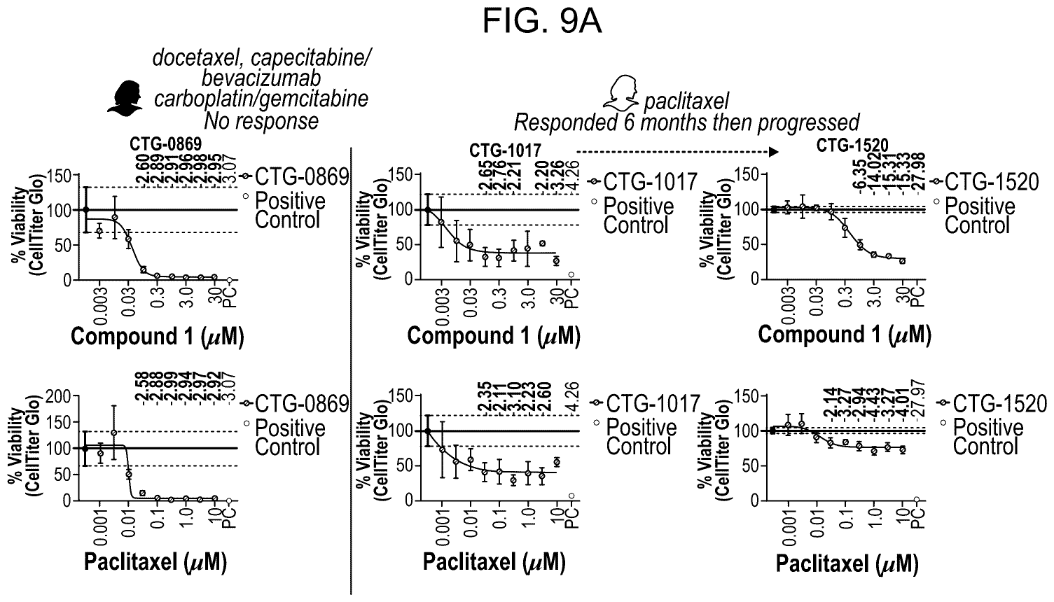

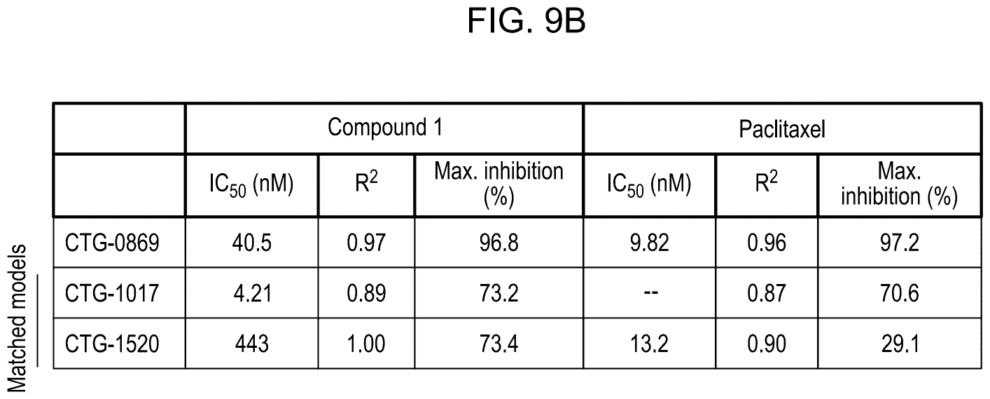

As shown in FIG. 9, Compound 1 was potent in all three (3) PDO models tested. Moreover, Compound 1 retained potency, when paclitaxel did not, in the matched PDO models derived from the same patient before (CTG-1017) and after (CTG-1520) taxane-based chemotherapy.

FIG. 10B shows the mean tumor volume of each group over time and the growth of tumor in each individual animal. Based on patient treatment refractory history, CTG-1520 is a paclitaxel resistant model which was confirmed ex vivo (max effect 29.1%) and in vivo (TGI 10%) (data from Champion Oncology Lumen Platform database.championsoncology.com/models/filter). As shown in FIG. 10B, Compound 1 treatment of CTG-1520 PDX tumors led to a significant tumor growth inhibition at the highest dose (150 mg/kg) throughout various days of the study. The 75 mg/kg dose of Compound 1 also reduced tumor volume relative to the vehicle control, but to a lesser extent than the 150 mg/kg dose. No body weight loss related to Compound 1 treatment was observed in this study (FIG. 10C.)

### Example 9. Investigation of Compound 1 Anti-Tumor Efficacy in Solid Tumor Cancer Cell Line Xenograft Models of Colorectal Cancer, Prostate Cancer, and Lung Adenocarcinoma

### Methods

Anti-tumor efficacy of Compound 1 was evaluated against three (3) different preclinical oncology mice models of colorectal cancer, prostate cancer, and lung adenocarcinoma (see Table 5 below). For this purpose, an oral formulation of Compound 1 was used.

TABLE 5
Compound 1 anti-tumor efficacy in pre-clinical solid tumor models

| | | | In vivo |
|---|---|---|---|
| | | In vitro | End of the study |
| Model | Cancer type | Maximum effect (%) | Maximum TGI (%) |
| COLO205 | Colorectal Cancer | 95 | 35.38 |
| | | | (with 150 mg/kg dose) |
| DU 145 | Prostate Cancer | 91 | 38 |
| | | | (with 150 mg/kg dose) |
| A549 | Lung Adenocarcinoma | Biphasic (52%/100%) | 34.64 |
| | | | (with 150 mg/kg dose) |

Human Colorectal Cancer Model—COLO205

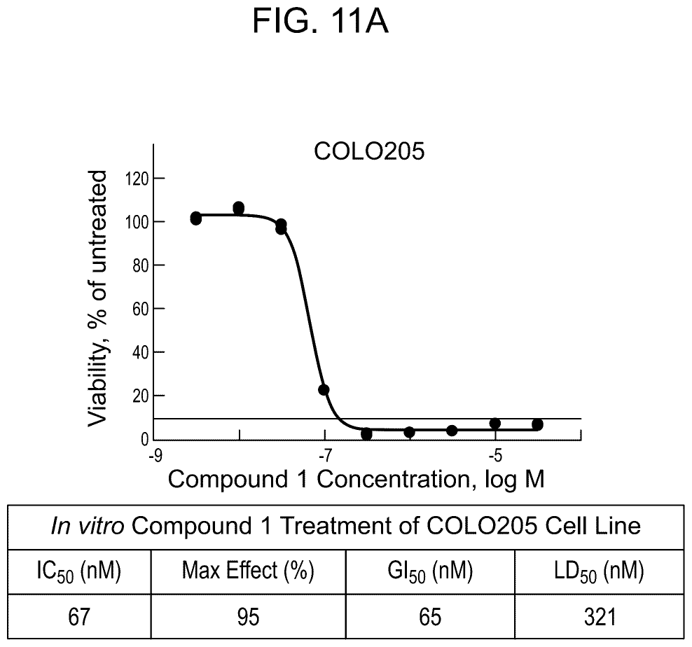

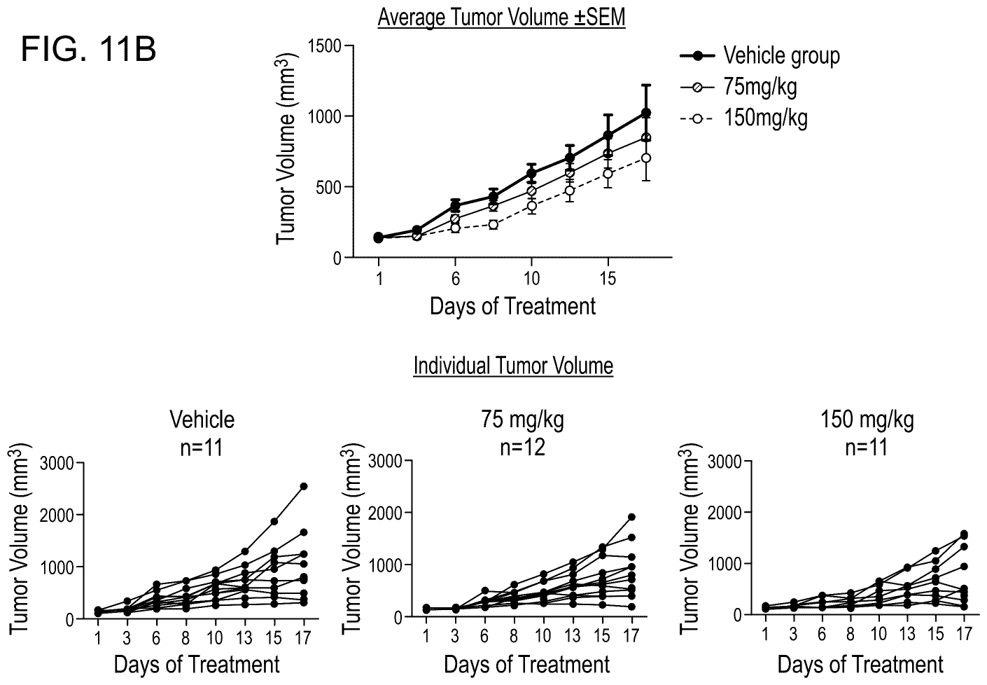

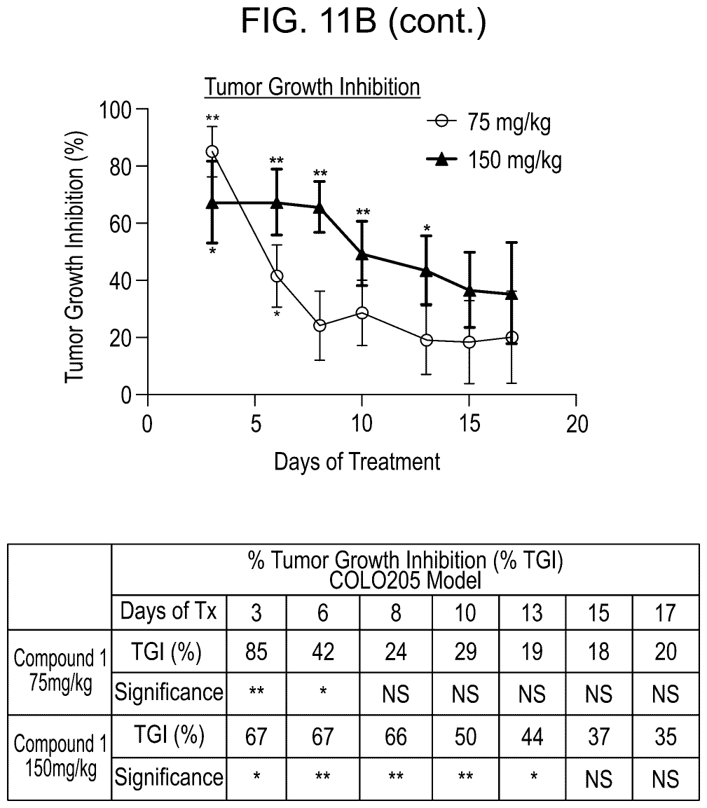

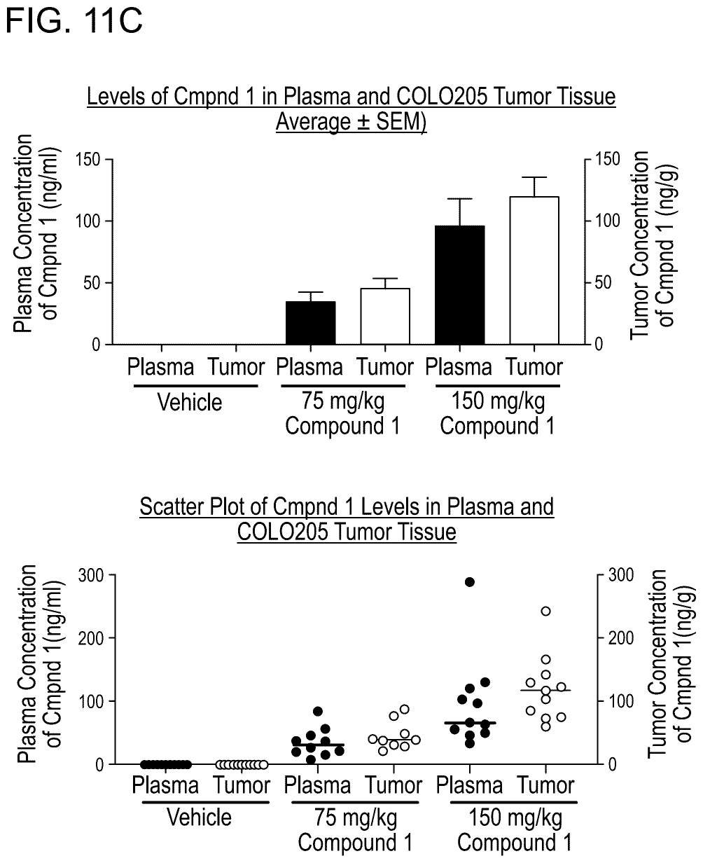

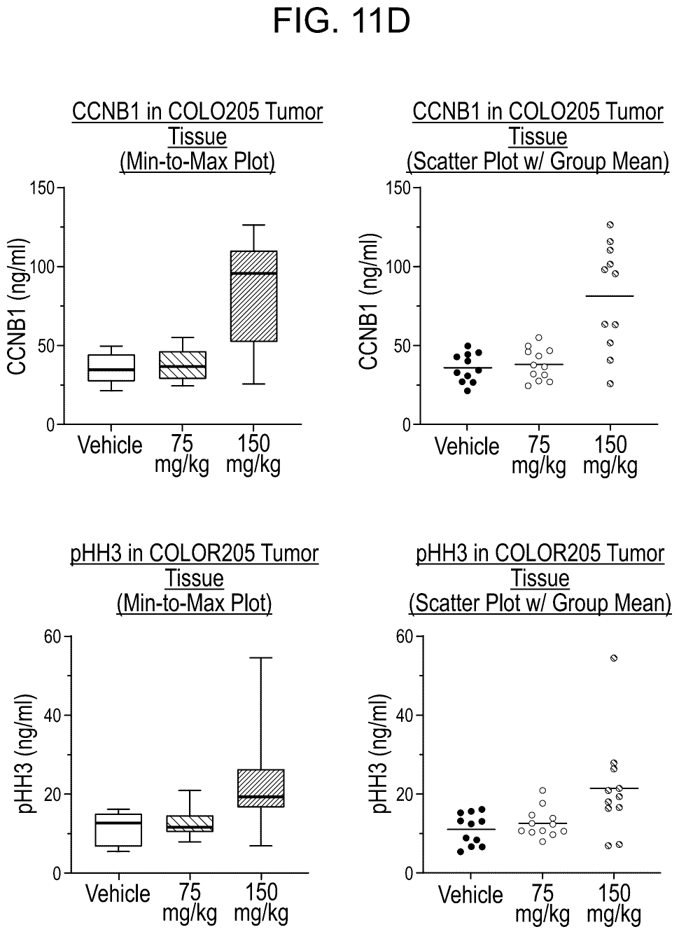

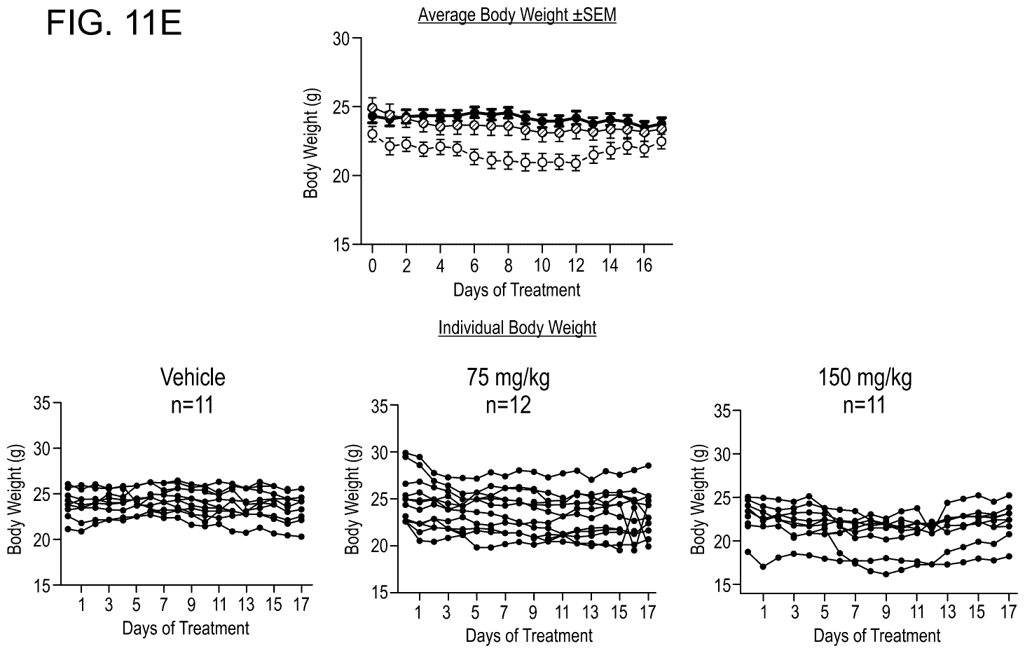

A previously performed screen of 102 cancer cell lines revealed that colorectal cancer cell lines were among the cancer indications with highest percentage of response and lowest IC50 for Compound 1. One of the most responsive cell lines was COLO205, which demonstrated a maximum effect of 95% and low IC50 of 67 nM (FIG. 11A). COLO205 is a colorectal adenocarcinoma (Dukes' Type D) cell line of epithelial origin isolated from human colon. COLO205 is a model that is well validated in the cancer field for in vivo efficacy evaluation of oncology therapeutics. Nude mice were implanted subcutaneously with COLO205 cells and tumors were grown to an average tumor volume of 113 mm³ followed by randomization into three (3) groups consisting of vehicle control, 75 mg/kg and 150 mg/kg Compound 1 (12 animals per group). A Compound 1 solid dispersion was administered by oral gavage two times a day (BID). Originally, the study was planned for 28 days of dosing; however, several animals left the study after 17 days of dosing due to tumor size reaching the limit of 2000 mm³. Therefore, only the data for the first 17 days of dosing were used for the analysis. Additionally, one animal from vehicle group and one animal from 150 mg/kg group left the study prior to day 17 and were therefore removed from the analysis. The percentage of tumor growth inhibition (TGI %) was calculated for each individual animal.

### Human Prostate Cancer Model—DU145

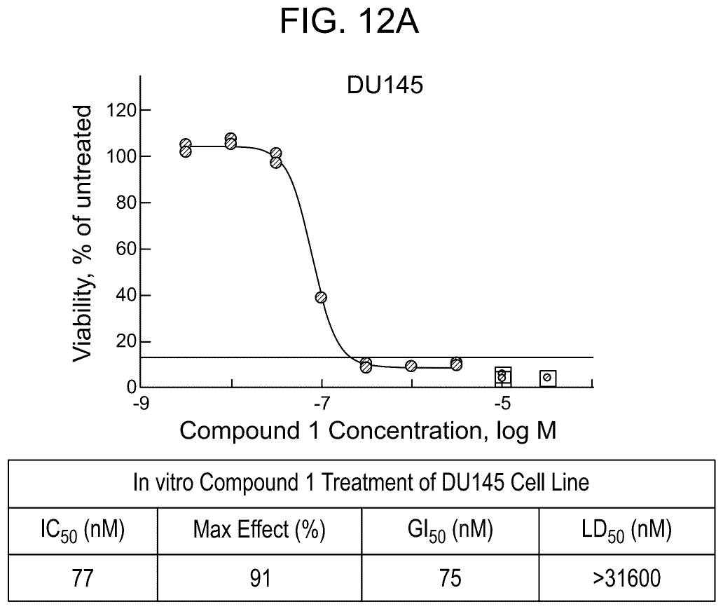

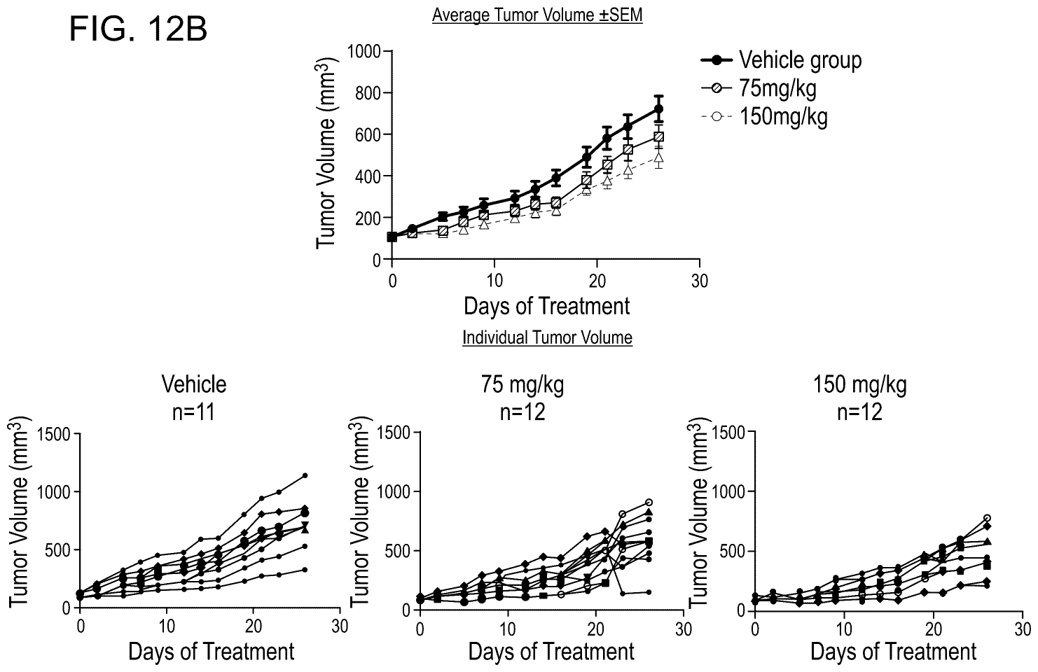

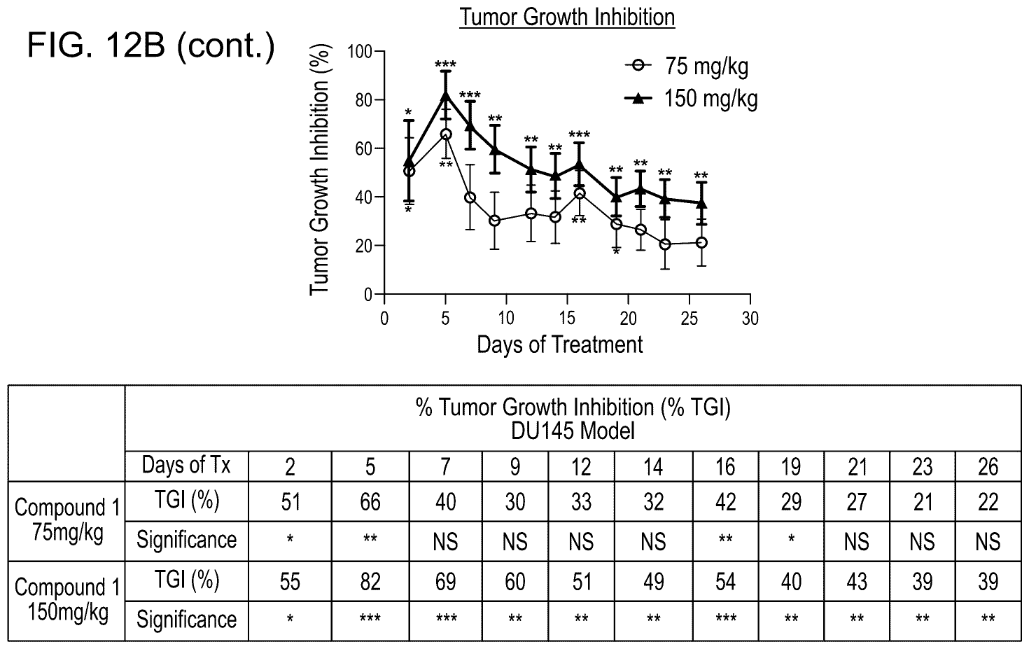

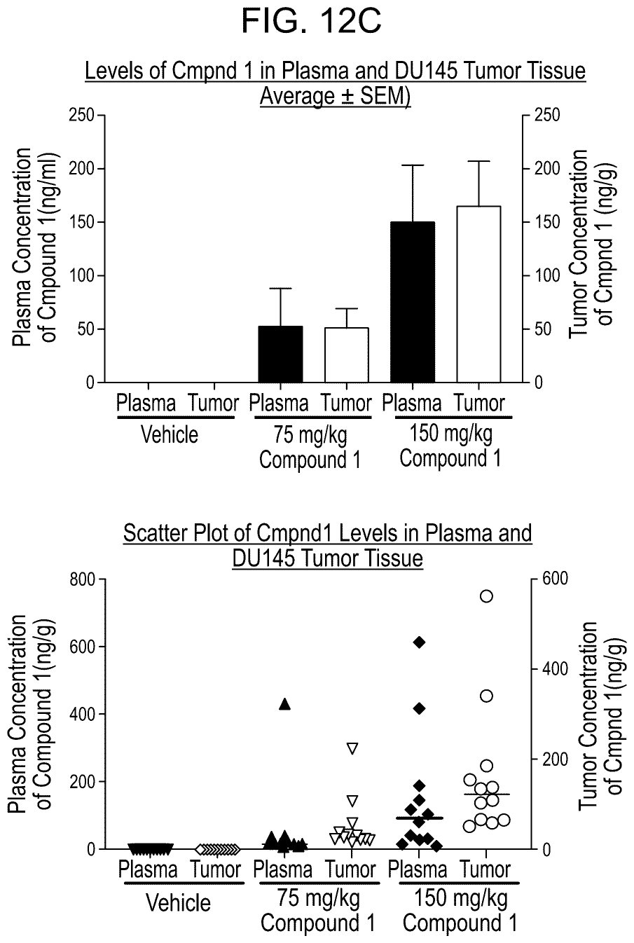

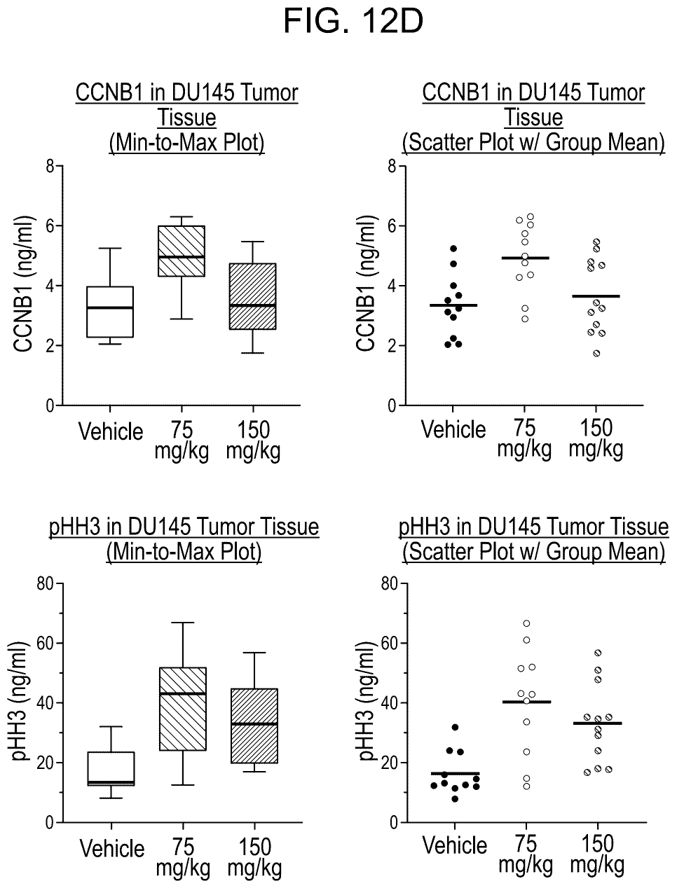

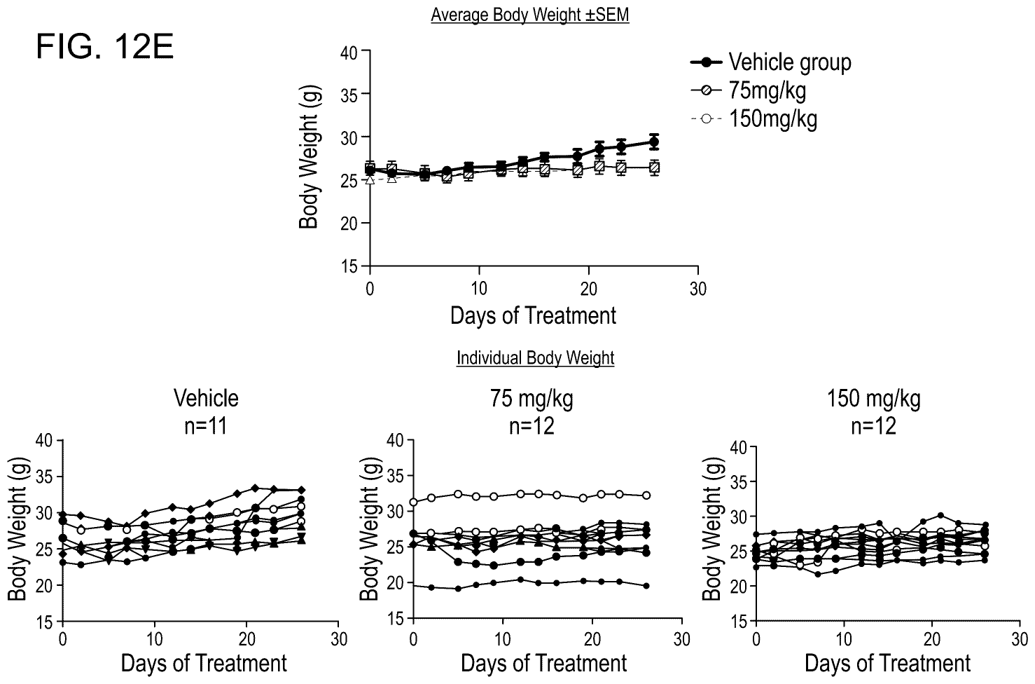

DU145 is a human prostate cancer cell line that responded well to Compound 1 in vitro with an IC50 response of 77 nM and maximum effect of 91% (FIG. 12A). DU145 is an epithelial cell carcinoma derived from the metastatic site (brain). DU145 cells were implanted subcutaneously in nude mice and tumors were allowed to grow to an average volume of 106 mm³ followed by randomization into three (3) groups consisting of vehicle control, 75 mg/kg and 150 mg/kg Compound 1 (12 animals per group). A Compound 1 solid dispersion was administered by oral gavage two times a day (BID). All animals, except one animal from vehicle group, survived throughout the 26 days of the study. Any animal that left the study prior to Day 26 was eliminated from all calculations. The percentage of tumor growth inhibition (TGI %) was calculated for each individual tumor. The mean tumor volume of each group over time and the growth of tumor in each individual animal is plotted below. The percentage of tumor growth inhibition (TGI %) was calculated for each individual animal.

### Human Lung Adenocarcinoma Model—A549

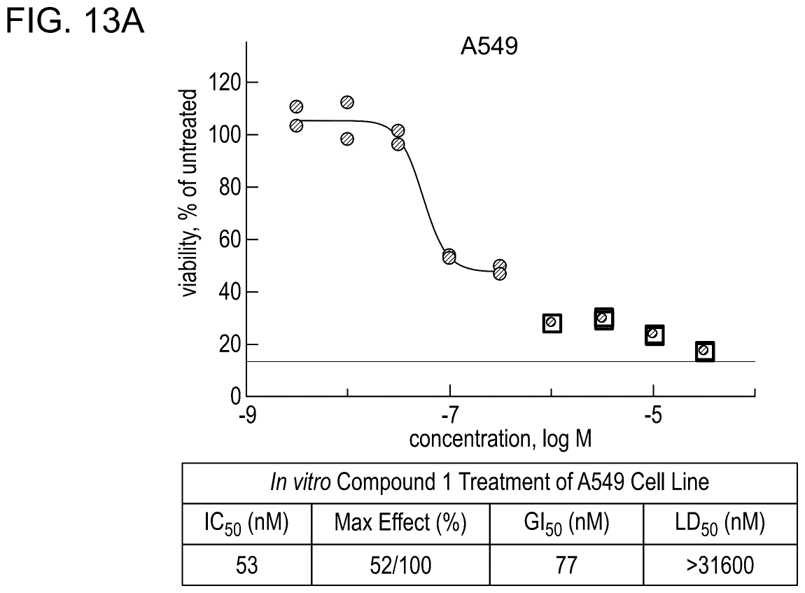

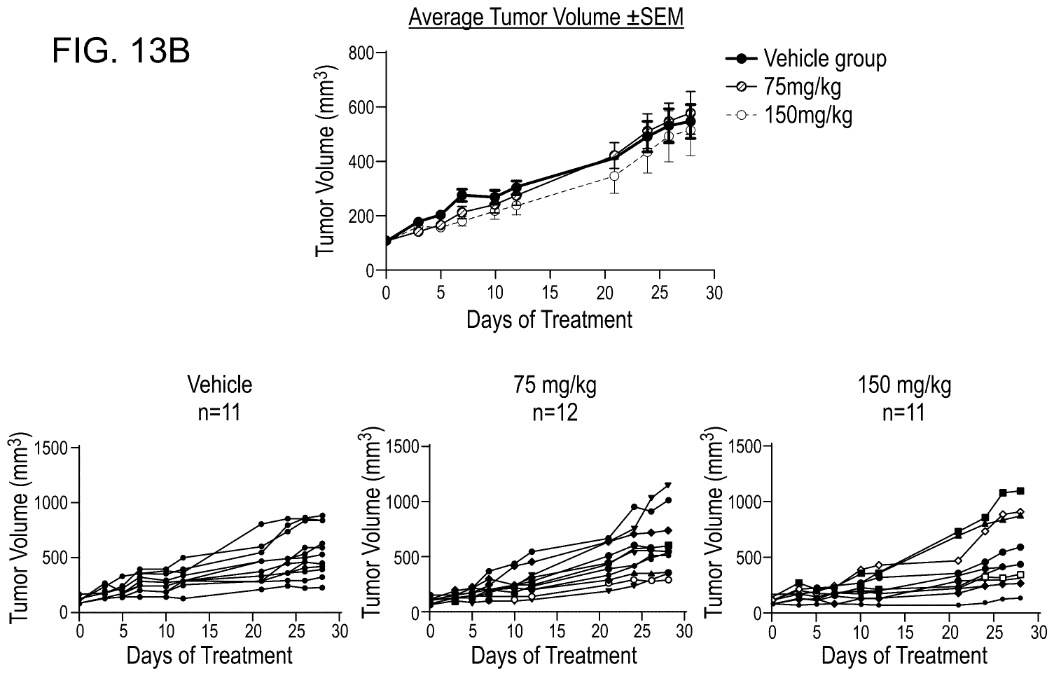

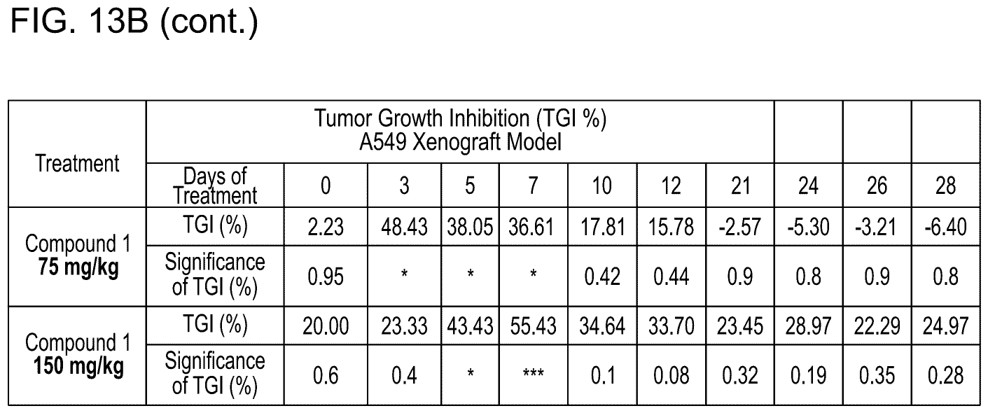

A549 is a human lung adenocarcinoma cell line isolated from the lung tissue of their origin. A549 cells demonstrated a biphasic response to Compound 1 in vitro with an IC50 response of 53 nM and maximum effect of 52% in first phase followed by 100% at highest concentration tested (FIG. 13A). A549 cells were implanted subcutaneously in nude mice and tumors were allowed to grow to an average volume of 112 mm³ followed by randomization into three (3) groups consisting of vehicle control, 75 mg/kg and 150 mg/kg Compound 1 (12 animals per group). A Compound 1 solid dispersion was administered by oral gavage two times a day (BID). All animals remained in the study until Day 28 except one animal from Group 3 (Animal #29), which exited the study on Day 11 due to more than 20% body weight loss. This animal was excluded from all analysis. Data from Day 14-18 were excluded from the analysis due to inconsistencies in data collection methods.

### Results

As shown in FIG. 11B, Compound 1 was able to inhibit the growth of COLO205 tumors in a dose-dependent manner (e.g., 20.13% at 75 mg/kg vs 35.38% at 150 mg/kg at day 17). This was further reflected in a dose-dependent elevation in the level of Compound 1 in plasma and tumor tissue (FIG. 11C) as well as an increase of cyclin B1 and phospho Histone H3 (pHH3), both markers of increased M phase arrest, in tumor tissue (FIG. 11D). Although lowest body weight was observed in the 150 mg/kg treatment group during the middle of the study, this effect was within a 20% variation of body and transient reflected by no significant body weight change by the end of the study (FIG. 11E). In conclusion, Compound 1 demonstrated efficacious potency in inhibiting the growth of COLO205 tumors.

As shown in FIG. 12B, in nude mice, Compound 1 was able to inhibit tumor growth of DU145 tumors in a dose-dependent manner (e.g., 22% with 75 mg/kg vs 38% with 150 mg/g at day 26). Significant tumor growth inhibition was observed, particularly with the highest dose, during various days of the study. Dose-dependent efficacy reflect the dose-dependent increase in the level of the drug in the plasma and tumor tissue as the dose of Compound 1 increases from 75 mg/kg to 150 mg/kg (FIG. 12C) and correlates with an increase in the level of two biomarkers, CCNB1 and pHH3 (FIG. 12D). Only one animal from the vehicle group exited the study early, subsequently data from this animal were eliminated from all calculations. There was no body weight loss related to the drug in this study indicating that the drug was well tolerated during the duration of the study (FIG. 12E). In conclusion, Compound 1 demonstrated efficacious potency in inhibiting the growth of DU145 tumors.

The data from the human lung adenocarcinoma (A549) study demonstrated that treatment with Compound 1 decreased the average tumor growth at all doses evaluated (FIG. 13B). The highest TGI (%) was observed on Day 7 of treatment with 36.61% at 75 mg/kg and 55.43% at 150 mg/kg, respectively. The data from the body weight measurements demonstrated no drug effect in any of the animals.

### Conclusion

Compound 1 demonstrated anti-tumor efficacy in various pre-clinical solid tumor models including colorectal, prostate and lung.

### Example 10. Investigation of Compound 1 Anti-Tumor Efficacy in an Animal Model of Glioblastoma

### Methods

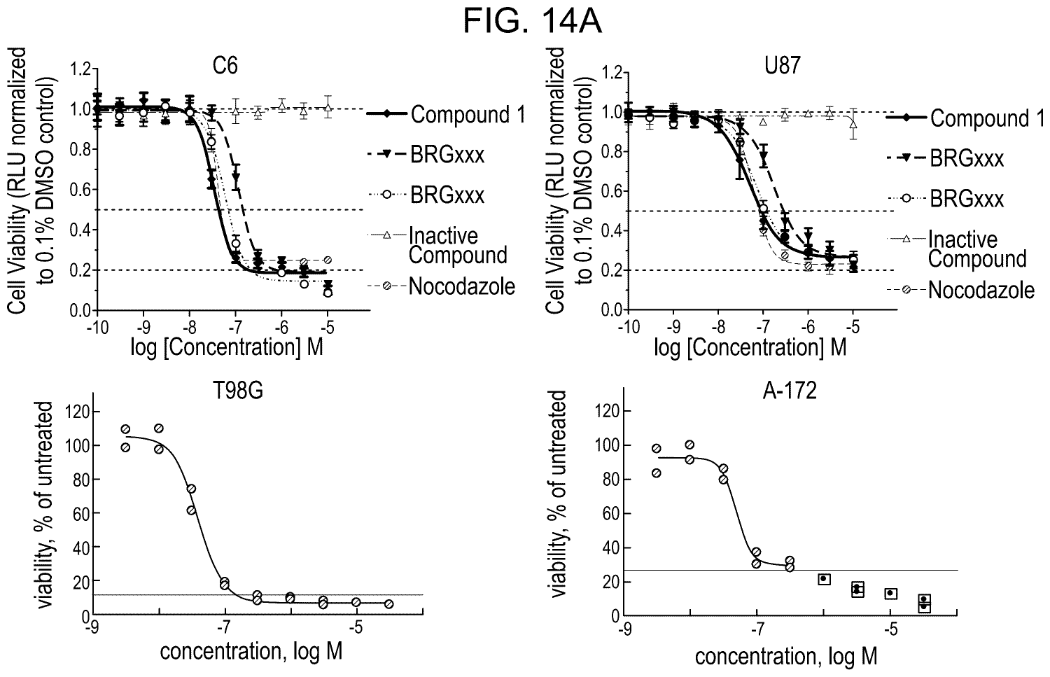

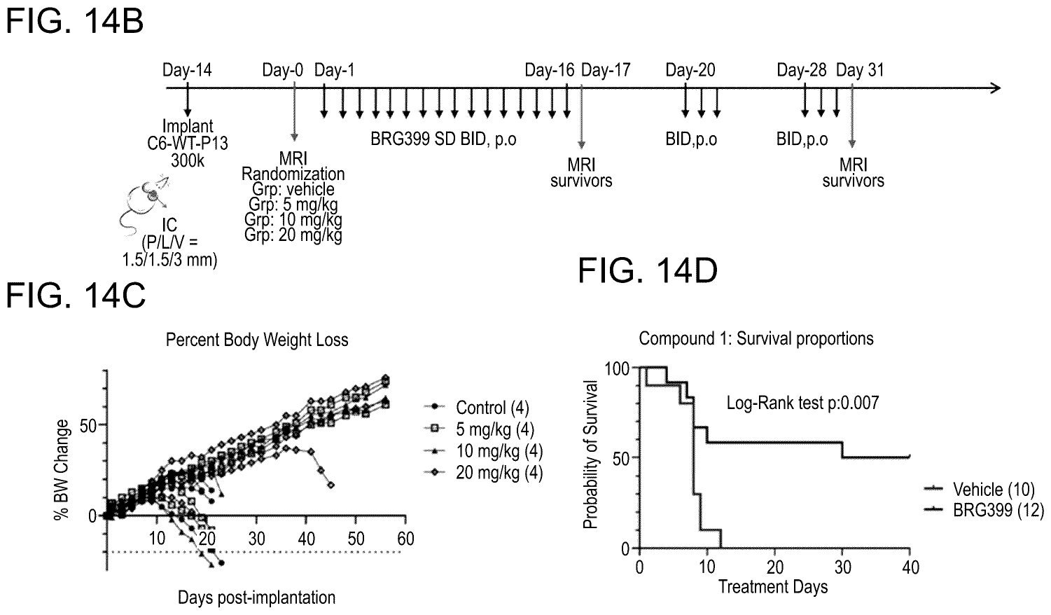

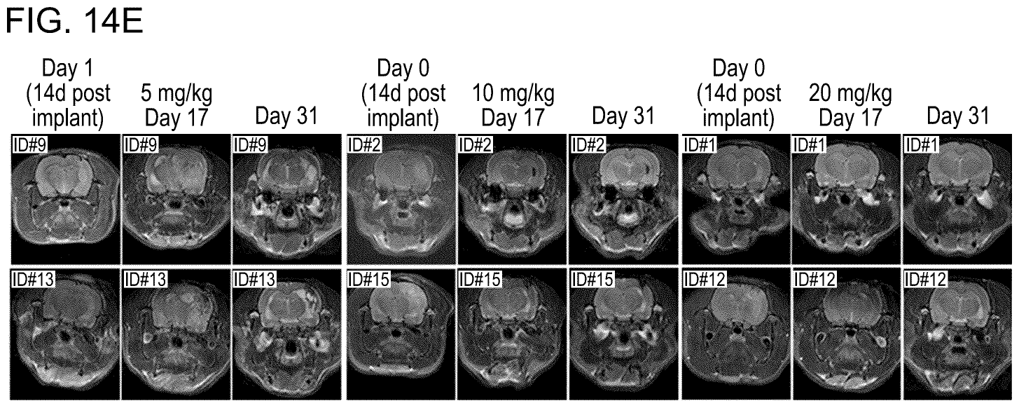

Glioblastoma (GBM) is the most common and highly malignant form of primary brain tumor with a poor prognosis despite advances in treatments. Therapy resistance and relapse remain a significant clinical challenge necessitating the development of novel therapeutic approaches that are not limited by the blood-tumor barrier. Accordingly, the in vitro potency of Compound 1 was examined in four different glioblastoma cell lines as evaluated by Realtime-Glo™ assay (C6, and U87 cells) or ATPlite 1Step™ Perkin Elmer (performed by NTRC Oncolines; T98G and A-172 cells). An inactive compound, BRG396, was used as a negative control, and nocodazole was used as the positive control. Nocodazole is known to be a mitotic agent that induces cell death. The rat C6 glioma cell line, in particular, is an intensively studied model used to evaluate therapeutic efficacy of a variety of modalities. Therefore, the in vivo tumor activity of Compound 1 was next assessed in Sprague Dawley rats implanted with C6 rat glioma cells into the right entorhinal cortex/subiculum region (AP: −7 mm; ML: 4.5 mm, DV: 5 mm). 14 days post implantation magnet resonance imaging (MRI) was performed to assure successful implantation and animals were randomized to receive either vehicle or Compound 1 treatment (5, 10, and 20 mg/kg groups) twice daily by oral gavage for 16 days (Day 30 post implantation). An overview of the study design is shown in FIG. 14B.

### Results

The anti-cancer activity of Compound 1 in the in vitro models of GBM was assessed to be in double digit nanomolar range (ranging from 42 nM to 89 nM) potency in four different cell lines tested:C6 cells: 42 nM, U87 cells: 89 nM, T98G cells: 39 nM, A-172 cells: 48 nM (FIG. 14A).

FIG. 14C shows the graphed bodyweight recorded every 2 days throughout the in vivo study. Serial MRI's were performed on survivors on day 17 and day 31 post implantation. As can be seen in FIG. 14D, Compound 1 administration resulted in a marked improvement in survival (from 7.7 t 2.6 days to 25.5 t 15.6 days, log-rank test, p:0.007). Moreover, six of the animals receiving Compound 1 treatment (2 in each treatment group) were still alive at day 60 post-treatment initiation. MRI imaging performed on the surviving animals shows reduction in tumor size and tumor fragmentation (FIG. 14E, top and bottom panels show images from two different animals). These results indicate a desirable therapeutic effect of Compound 1 on intracranial glioma-bearing rats, demonstrating the therapeutic potential for Compound 1 in the treatment of GBM.

### Conclusion

Compound 1 demonstrated efficacy in treating glioma in an orthotopic syngeneic glioma model.

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating a cancer responsive to the modulation of microtubule assembly comprising administering to a subject in need thereof, a therapeutically effective amount of 2-(difluoromethoxy)-N-[[5-(2-methoxyphenyl)-1H-1,2,4-triazol-3-yl]methyl]benzamide, or a pharmaceutically acceptable salt thereof.

2. The method of claim 1, wherein the cancer is selected from prostate cancer, head and neck cancer, endometrial cancer, glioblastoma multiforme, sarcoma, and lung cancer.

3. The method of claim 1, wherein the cancer is prostate cancer.

4. The method of claim 1, wherein the cancer is head and neck cancer.

5. The method of claim 1, wherein the cancer is lung cancer.

6. The method of claim 5, wherein the lung cancer is small cell lung cancer (SCLC) or non-small cell lung cancer (NSCLC).

7. The method of claim 1, wherein the cancer is endometrial cancer.

8. The method of claim 1, wherein the cancer is glioblastoma multiforme.

9. The method of claim 1, wherein the cancer is sarcoma.

10. A method of treating a P-gp-mediated resistant cancer comprising administering to a subject in need thereof, a therapeutically effective amount of 2-(difluoromethoxy)-N-[[5-(2-methoxyphenyl)-1H-1,2,4-triazol-3-yl]methyl]benzamide, or a pharmaceutically acceptable salt thereof.

11. The method of claim 10, wherein the cancer is a taxane resistant cancer.

12. The method of claim 10, wherein the cancer is resistant to paclitaxel, vinblastine, vincristine, or docetaxel.

13. The method of claim 10, wherein the cancer is resistant to paclitaxel.

14. The method of claim 10, wherein the cancer is selected from ovarian, prostate, breast, bladder, head and neck, and lung cancer.

15. The method of claim 10, wherein the cancer is ovarian cancer.

16. The method of claim 10, wherein the cancer is breast cancer.

17. The method of claim 10, wherein the cancer is bladder cancer.

18. The method of claim 10, wherein the cancer is head and neck cancer.

19. The method of claim 10, wherein the cancer is lung cancer.

20. The method of claim 10, wherein the cancer is a vinca alkaloid resistant cancer.

21. The method of claim 10, wherein the cancer is resistant to vinblastine or vincristine.

22. The method of claim 10, wherein the cancer is selected from lymphoma, acute lymphocytic leukemia (ALL), and solid tumors.
